SEQUENCE LISTING (8 sequences)

SEQ ID NO: 1 - protein, 1306 aa, Human
MGAASGRRGPGLLLPLPLLLLLPPQPALALDPGLQPGNFSADEAGAQLFAQSYNSSAEQVLFQSVAASWAHDTNITAENARRQEEAALLSQEFAEAWGQKAKELYEPIWQNFTDPQLRRIIGAVRTLGSANLPLAKRQQYNALLSNMSRIYSTAKVCLPNKTATCWSLDPDLTNILASSRSYAMLLFAWEGWHNAAGIPLKPLYEDFTALSNEAYKQDGFTDTGAYWRSWYNSPTFEDDLEHLYQQLEPLYLNLHAFVRRALHRRYGDRYINLRGPIPAHLLGDMWAQSWENIYDMVVPFPDKPNLDVTSTMLQQGWNATHMFRVAEEFFTSLELSPMPPEFWEGSMLEKPADGREVVCHASAWDFYNRKDFRIKQCTRVTMDQLSTVHHEMGHIQYYLQYKDLPVSLRRGANPGFHEAIGDVLALSVSTPEHLHKIGLLDRVTNDTESDINYLLKMALEKIAFLPFGYLVDQWRWGVFSGRTPPSRYNFDWWYLRTKYQGICPPVTRNETHFDAGAKFHVPNVTPYIRYFVSFVLQFQFHEALCKEAGYEGPLHQCDIYRSTKAGAKLRKVLQAGSSRPWQEVLKDMVGLDALDAQPLLKYFQPVTQWLQEQNQQNGEVLGWPEYQWHPPLPDNYPEGIDLVTDEAEASKFVEEYDRTSQVVWNEYAEANWNYNTNITTETSKILLQKNMQIANHTLKYGTQARKFDVNQLQNTTIKRIIKKVQDLERAALPAQELEEYNKILLDMETTYSVATVCHPNGSCLQLEPDLTNVMATSRKYEDLLWAWEGWRDKAGRAILQFYPKYVELINQAARLNGYVDAGDSWRSMYETPSLEQDLERLFQELQPLYLNLHAYVRRALHRHYGAQHINLEGPIPAHLLGNMWAQTWSNIYDLVVPFPSAPSMDTTEAMLKQGWTPRRMFKEADDFFTSLGLLPVPPEFWNKSMLEKPTDGREVVCHASAWDFYNGKDFRIKQCTTVNLEDLVVAHHEMGHIQYFMQYKDLPVALREGANPGFHEAIGDVLALSVSTPKHLHSLNLLSSEGGSDEHDINFLMKMALDKIAFIPFSYLVDQWRWRVFDGSITKENYNQEWWSLRLKYQGLCPPVPRTQGDFDPGAKFHIPSSVPYIRYFVSFIIQFQFHEALCQAAGHTGPLHKCDIYQSKEAGQRLATAMKLGFSRPWPEAMQLITGQPNMSASAMLSYFKPLLDWLRTENELHGEKLGWPQYNWTPNSARSEGPLPDSGRVSFLGLDLDAQQARVGQWLLLFLGIALLVATLGLSQRLFSIRHRSLHRHSHGPQFGSEVELRHS

SEQ ID NO: 2 - DNA, 7276 bp, Mouse
  misc_feature 7216: n = A,T,C or G
acgcgtgctagtctttgggttggaattgtattgacacacagtttgccgtcttgaaatcatgactgtcttctaatgaaatctttttcattaaggtcttctgaacttcaggcctgtttcatgagtcggtatgtaagtcctgcacttcctcagttacatttattcccaactgtaaaaagatttgttttatttagttagtatttttcaagacttcatttctccgggtagccttggctgtcctaaaactcactgtgaaggcctctgcctaccacgtgctggggttaaagagtgtgtgtcaccaccacctgggtagttacgcttatttacttgtttgtttatgtgtttatttgtttatgtgtgtgttggtgcatgcatgtgtggaagttaaaggacaatttgcaagagtcagttctctccattcaggtcacccagaagatgaaaacttcttaatttaaaacaccgggtcatcggcttggtagcaagccccatttatctaatgagccacattaccagctttacttgaaattaatttttgatgctatgataaggtgtgctggagagatagatggctctggttaaagagagaagactggctttgcaaaagacccgaacccagttcctggcacccacgttgggtggctcagaagtcacctgtaactccagctcccggagactcagtgcctctggcttctgtgggcacctgcactcacttacacgcaggtctacacacacgtgtataactaaaaataagtcctaaaaatgaaaacttttattttccttttttgatcactgctagtagacagacatacaacttagttttgagtgctgagcttgtattctgtgggtttatttaattcaattattatatctaatagttggttttgtagtttattaagatatatatatatatatatatatatatatatatatatatattctttttcattaagagttcctctgtctcttttccttcctggttactctggctaaaatttcctacacaatgttaaaggggggaggggggagggagaggaagaggggaggggacagggagggggaggatgcatccatgatctaagaggaagcgctttctgtccagccacaggatgagctatgcctttatgaaaaagcccagaatcatgctgagggtatattccctttcactccatcttggttgaactttttaaaattttgaatgtatgttggatttttgtcaacacccctggggacgctcacctcggactgtgtgtttgctctattggtatggtgtgctccactgagcttttctgaatgaaatcgtaaaagaaccagctaaaacacacaccatcaactgtctttaaaaagcgagggatgtcctgggaggtggcttaatcagtaaagtgcttgcaacacatgcatgaggttctgagttcggatcccagcaacccggtaaatgccatgcctggtaggtaacacatacccataactttcgctctgcagaggtcaagataggaggatttctggggctttctggtcagctagtctagccactcatgtgaagaggcactgaggcagacacccagaattgatctctgctgtccactcgcatgcctagtcactctcacgtactgccacacatacacacacctatgtgtccatatatgaacacagacacacatatatcacacatagagaaagaagaaggcaagggaatgatcttttaaaaatagaagtcaagtgtaacagatttcttgttaaaaataaccacagacagttctacacccttacctcattggtagccgtgcgaccatgaacaacctcacaggtatttgacctctcctccctcgggctctccatatgcaaaatgggaatgaaatcagagcttacctctgagaatatagaaatccacacaccccggggtctccggtttcctccatcaacagagaagaatctgtaaccaaatgtgtgtggtgggagtggattatgcgtcacagcaaacggactatgcattctgcagtgcacgccggctgagtgtcttctaattctgtctgatgctgacaccatctacgtggagagagctcatgtctaagttaggaacccagcctccatttcactccacttactgattaggggaccagaatcctccatgggtctcattaattcactagagtagctcactttacgtgtgaaccttttaaacgcattacacgtatgtgcaccacgcgcatgcctgttggatgccctagggttatggatggccaccatgtgggtgatgggattcgaacctaggtcctctgcaagaccaatatgtactcttaaccactgagtcatctttccagcctcctactgatttgttacacaggacatgacaaagggctagagatccccaaatgaagatctggggacaaggggacataccctcctctaggaatgtctactttaaccagaaacttctagtcttccttagtctttcaggtaactccactctatcctgaagcctcctaacagctgtcagctgccagtcaactcaactcgatggttcataaaagggacagctttgaagagtacaaagattctgggggttgtgtctcagcaaacacatgctacatagtatcattaccagaatcctagacaaccaggacgaatctcagcaagtcagggtctgaccacttgccctagaaatcaactatgaaaagtgattgctagaggcaggaacagacttgaagcgtccaaggccctgtctctggggaagagttcaggtttaactataagaagacctggttcagcggggcaagagtcctgctcacagtgggtgctgttggattttaccagttggtcccagaggagttattcctggctttcatggcttgaggaaacagtttggtccccatggctcatatttaggggtaacttcatttcttctcatggggtgaccgctctcacctagggggataatgtcatcatggagtgatttgtctacaggacttgttatttttggaacctagaataagtttggtttaggacaattagtggagacttccagttgctgctataggaggctcattactgtcaaagtccaaatctccagttaccttgaggccttctgccttaagggcagatgagaaaggtatgaagaatgtaggataacctgacctctctcagccaggctcactgctcatctgggagactcttatttatgctcctgactcagaggctgctgctcttgggtatgactcctctcctaagtgtccttagaggtggtttcctaagtctctcaaactccatcatctcccttcaggatcagttgagcctggccccagattctgcctcttcctctggtgtgtggtgtgtgtgtgtgtgtgtgtgtgtgtgtgtgtgtgtatgtgtgtgtgctttgttttcttctagagacccaatatttccaaattctgtagttccctttcaggcaacctaaaaaaaaaaaaaaaaaaaaaaaaggggaagaggagccagtgcaacagacaggaacgtgttcatctgttcccgtcctcacagaactagcagctgggagcccctcgcccagccgactctccaacctgcatcggctcacgctatcccctggaggctatggagttggggcctcctctggtcctgctgctggccacagtttggcatggtaaggggagaaaaggggagtcctgctgggggagtgttgcaaagagggcatcgctgtcctgcagtagatgcctcattctctgcttcacttctctggcataagagtgcagatttgtgtttatctgtcgagaagaggtaggtgagagtgggtggaaccacccggggccagtgctgtggatgtgtctgagcctaggctttgactcacggtgtgctctgagtgtgaccctggtattttcaggaccaccatccacatctactcgtctgcaggtgggagggagggcggtagtgggctaaccttgagtgcagcgctttggggtgttacctgggagtctcctttaaggcagattcccataggtcttggtcaggactgagattcgcatttctgaggaaatcttatgcgtcagggctgctcaatagcttttgctgttcttctttgagtggtcccaccagctggacagcccagcaggctgtggttaattaggacctggcccattcttggaaggcttgggtgaaaagggtcagggtagaggaaagaagaggcctttttggcccaggactcaagggtgaaaagtttggacaggggtcggaggagaacacccgtgagtgagagatcctatgaataagcaaggaaggatgctagagaggggcctggggaaggccagagagggtctccctagacactgtcttcctaggtctctccccccaacttctcgggcagcagaagctgcttcggaactcccattcttggaggaggtctggctgagggtgagaacgaacataaatggcccagtgcagcgcaaggatcccatctaggctccagctggcctagcacacacatgttctataccacaccactgcttatttggaaagaccttgagggaagatgctggggaaggacaggctcattctggtccctggggaattggtgagatgacagtccgtggaaggctctttgctggctcagccgatctcactggctcgtgcactggcacattcctagtagaatgggattggtatgtatgcatgagaaagcacacattcttaagaatactgttaaagggcatgtgacagatgtgtgtgtgtcaatgcagccctgactcagctaagaaagagacaattattattcctctcatgccagaaaatcctgtgatttttggttcactgcttttcagtctttatctcgagggctgcttgtggggcagagtgtgtgagtggctgcatgcctgcgtgtgtgtgtgtgtgtgtgtgtgtgtctgtctgtctgtatccagctccaaaaggaagagaagttgggagatatcagtggaaagcctggggttttaacctggaaaattattggtgtgtgggtgggaccatgggggcttctgtaggagctggaatagtgttgaagcatttagtggcagagagagagaaagagagagagagaaagagagagagagagagagagagagagagagagagagagactggcctcttctctttgagtggtagtcatgagtgcaagaggttggggagcctcctggagctgatggtggaagagtccattttgcaagtacaaagaggagaaagtttgggtcatggggagaattccagaagggagatgcgagatgaacaatggccaggatcagggcatcagtggccatcaagtgctcatccaagggaaggtagtgatcagcgtgcttccctcccgccaaccctgccagcgggaagtgagaccatcatgcggcttacgtggaattcgtcagccctgttctgtgtaaatatccagggcaacaggctaggcagaaataagaaggaagcttctctcggagcacatttcctggaggccaagacaggagacaaagataagcagagcagagtagcccgggagaagggagaagtgtctggaacccaaagatgtgtcctgtgacctttagggttaaggtccttagctaccacatagcaggcccaagagggacattagcagtacggcctgagaacagcaggtgggtcaggaggactccactgccctctaggtggccgctgggattgttctcacaggacagaagaatatctgatgctcagagggtcaatggctatcagggtccctagccatagtcaatgtgagcccagggtgaaatagctcgtccctcttcctgctgatcactgcccaggagagccagagccctacatcagcagcagcacactgctctcctctgttccacatctggctggttggaggcttgggcagacaggccacatgtttccaaagagtttgaatcagagtgagcagacattaaaccaggctgaaatcatctgtgagatgagactttccatttctgggctcgtgtgtgtgtgtgtgtgtgtgtgtgtgtgtgtgtgtgtgtgtgtgacccactgagagcggttgtaaggaaaccctgaagtcctctaagggcttgatgcccttagggctgccctgtcactgtgtaggaagggttgacttgcggggtcagcaaacaggacagtggcacagcacagacagactgtgcccagtgtgctctgaaacccagcactccttgccaagagtccctcagtgtgtgagaaggacaatggccaggaggccagggaagcagaagtgagagcccaagtgttgaagggccacaggctgggcggaaaccagggccagccgggtgccagcaatgtgtttccgcccacacaggccgggggcgcctgccaggccctcagaggctgtgaatcagttctcacttccccccttcccccctatttcaagcctgggaaaaatgctgacaccacacaggcaacgagcctccttccctaagacctgacagggggtttgagttctcctttcgccttcaagcaaattttccttaaaagagattggctgccaagaaacccagcaggctcagagaagcgagggcagactgggtacctcagcagactgggtacctctctccttcatgagcatggcattctgagcccccaatgagtctgtactggagcaggggatgacacaacggtttcctggcatctgaacagcttagcatggcggggcaatgtgctcactgtctgcacttgtgggtgaaaagacaaaagggaggaggaggatggatggtctcaggccaccccacagcgcacagacccgatgcccagtggtaggagtgggggcagcaggagacacttggaagatctctttctactgggctaaaaactcatgtgtctttttttttttttyctttcccaagtgaaggaagatggcccaggacttggccatctgtttctccattggcttatttgaaacatgttcttacttaaacaggcttggcctgcttaktacacrarattgatggtgccctctcctgggatgggaaacratgccaaaggctgggccttccgtggctttgtggaraascctacatgtgtggctaaggaggcgcccacktaggtctcagggaatrascctcctgararttggcaratgtcaatarakttggaagctgattgaagggtccagactcattccagaaccagagccagagctatcccttccaaagcatggtccagtgttgggggacatatggggtgcaccccaggcctcacttgtgtntgccacctccttcaggtcagggggcccaggcgccgaccgtggtaccgagctcttacgcgt

SEQ ID NO: 3 - DNA, 8674 bp, Mouse
gcgcgcaccgcgccatgggggccgcgtccggccagcgggggcggtggccgttgtcgcctccgctcttgatgctgtcgctgctggtgctgttgctgcagccgtcgcccgccccggcactcgaccctggattgcagccgggcaacttttccccggacgaggcaggggcgcagcttttcgctgaaagctataactcgagtgccgaggtggtgatgttccagagcaccgtggccagttgggcgcacgacaccaacatcacggaggagaacgcgcgacgccaggtgggtgcccgggcccgggcgggggcggggcgcagcaggacgcagcggccaatcacaggcctgcggtccgtcgcctagggcggacagcagacgccagaacccccccgccccccagcctcaaccctgagcactgaggtccctaatgggacccctgccattaggccggaccctcccacccgctctgcgcattagacaggcaatgatgctccagtacgggctgggcacgtgatccaagccaacacaggaatggagtggatgaagctggctcctcagaggccaaaatcctcccgaagcggcatcaagggttggggcgatggctgtctgcgtacagggtacccagcttggctgcagagcccttgccctctggcatttgcctgatctcctgttgagtttctttccctggccctccactcactgtcccctactttggccccaattttctccttcattgttccctttgaactagattcccacactttccaaccagagggctcaggaggaggggtgctgcccctgcaagctcatccccaggacaggatctctgcaggaacaaggggtccggaagcccttagcctaccactgctctctcagttctgtagtctctcctacctgagcctctttctaagctgggctactctttctaccctcctggtgagcacaggaggaagcggccctggtcagccaggagtttgcagaggtctggggcaagaaggccaaggagttgtatgagtccatttggcagaactttactgactcaaagctgcgaaggatcatcggatctattcggaccctaggacctgccaatctgcccctggcccagcggcagcaggtgggtctgagggtggaggcagaacttgagctccccgggtgcctattgtggttgggaaagcagcccggcagaccggaagggactgatagtctgtttggaccttggctatcaggaatctggggagacagagtgctgttcatgggcttttccctccagatgcatggtgtgacaagtcactttgtattgtggagtagaaagccaaacgcgggagctggcgggtagctgaactagtgtgaggccctggagttcatactcagaattgtattgcacatacaaagagagatgtgctctctctctctctctctctctctctctctctcacacacacacacacacacacacacacacacacacagcacaaaggccaggggatagcagggattcatgcccgctgtgagctggagaccagctgatgcttgcctcggctctctggtcagtgggtaccacctgcaaaggctgcttgcactgaaaggggcaccagtatagtaggggttggaagaactgggtggccttgtctgagtcacatccttcctccaggccgctttttccttccagccaaaggggtgggaagattaactccaggggctaatatttggtcactaatatttcctcaactccaacttggcagtacaactctctgctaagcaacatgagcagaatctactccactggcaaggtctgcttccccaacaagactgccacctgctggtccctggacccaggtatggtcctgtccaatctctgctggggtttcctggtccttttgggtcacctctaggatgatgtctgtcagtaaaccaaaggcactggtaccccattggctggaatgtgcagatgtacccacacttctgcctcacacccttgctgcttctggcaaactcacttcagggagcctctagcacagcggttcttatcctgtgggttgtgacccttttgggggtcaaaacaacccttccacaggggtcacctaagaccatatttacccaacacagatatttacactatgattcacaacagtagcacaattacagacgtaaagtaacaacagaaataattttatggttgagggtccccacatgaggaactgtataaatggtggaggcattaggaaggttgagaaccactgggctagcccacccctccaccaccgtctggccacctagctctccttttaggggtcctttcaccttccccaaaagcttcccacaagccctgggtcccttggtgcttcgctgttttatgcaatgggccatggagggtttgtctattctaacctctaaatgtcctttctgtgtcaactgccttagacgttttcctgggagcagggtcctgcctggtgctttctttggatagcctcatctccccaaacccataggctgcccttggtctggggatactgtgatgcgtgaggaagctggtgggaatgaagaccagtgtatacctctggtttcataactgatcctgctcacctctctcagagctcaccaacatcctggcttcctcacgaagctatgccaagttgttgtttgcctgggagggctggcatgatgctgtgggtatcccactgaaacccctctatcaagactttactgccatcagtaacgaagcctacagacaagacggtgagcctgcatctttctctccatggacagacagccacctttcctatcttataaggcatccttgtctgctgtccccagtgagccctaaacccctgagctagataaaacagccagactgatggtctcaagatctcatagctagtgggaatggtatgaaacctgcagccaacagaccctgctgtcctgtcccttttactgatgctcctggtactacctgtggcactgctcctggccctaagactctctacctctttgtgttcttggacctgttttgctggagcctagagtggaccttgatatggtgacccatatctgtaatcccagtatttgaaaggctgagttagaaggatgaggagtttgaagccagcctggactacatagtgagaccctggctttgaaatgagggaggggggactagttatggcacacaactgtaatcccagagctggagagactgaggcaggaagatcttgagttcaaggccagcctgagcaagtctcactccagtgcttggaatcaggacaatggtagagttgaagctaggatgtggacagccaaagctctgggcctgacctgcactctcccaccctgttagacttctcagacacaggagccttctggcgctcctggtatgagtccccctcctttgaggagagtctggaacatatctaccaccaactagagcccctctacctgaacctccatgcctacgtccgccgcgcactgcaccgccgctatggggacaaatacgtcaatctcagggggcctattcctgcccatttgctgggtaagaacctgaaccttgctgcccagaggagctcgggtgcaggaggatgcctagaagagggacgatgggtcaccctcacagtgcctcaggcatgtggaatgtcataacctttgcttccttacacggaagccccccatggctcaatgtccccaccaagctggaatcctgctttagagactgaggtttatttagagccaagaatttagagccaatggaaaactggaatagagcagagctaggggtcaagtcgatggctttggcttcagggtcagggttagcagcagccttattgtggaccatacctgaggacatgagtctgggagcctggaagcctggaagcctgtggccccttagtgaaggatcctggcataatcggaaaagacaccagtaaccagtcctgtggagcccacagagtcaggggacatgtcctttggcccaagggtatataggtgccgacacccacacaggatagcatacataatgggaatggttgagcaggagaccaaactgtcccacttgcttcttccctgtaggagacatgtgggcccagagctgggagaacatctacgacatggtagtgcctttcccagacaaacccaacctcgatgtcaccagtacaatggtacagaaggtaggtcaatgagacaggagttgggggtcatgtgataggcatggggcccctccccttttctgcaatagtcaacagtgacctaaggtggcagagtgatctgctgtggtcagtagtgccctgcctttgggaagtgaggccactgagcctcagatacctttcctgtcctgggtggaagtcacaagccctgagctcaaggcatggctccatacggctgcctcctgacctgtttccctgcctgctggccagggctggaacgccacacacatgttccgggtatcagaggaattcttcacctcgctggggctctcacccatgcctcctgagttctgggcggagtcaatgctggagaaaccaacggatggacgggaggtggtgtgccacgcctctgcctgggacttctacaaccggaaggacttccggtcagacctggggagaggagtgcacaaggggtctaagggaggcggctgtgacacagacctgcctctgtactgggggattggtctatgccaagacaataagccacattagctcagtatccctgccgcttggagggacgaccagctgtgttgccttggcgtggcttgagtgatggtggatcagggtagggtcaatagggttgctagctcctgaactctcctcaccccagggcagagtcctccttggccctgtgttcctccaggaccagccttcccagcactctggtcctgccctcaacatcaccttcatccccatcatccccactccaggattaagcaatgcacacgggtcacgatggaacagctggccacagtacaccacgagatgggccacgtgcagtactacctccagtacaaggacctgcacgtctctctgcgtagaggtgccaaccctggcttccatgaggccattggggatgtgcttgcactctccgtctctacccctgcacatctgcacaaaatcggcctactggaccatgttaccaatgacataggtatggcgggggggggggggggggaggcatatccagcctggctcattcaaatctcctaccacagtagatccccactgctcctcactccatctcgtccttatttttaacttgagacaccagatgcacacaccctgaccttagcactcacccagagtccttgtctaagcggggcttagaacttgaggctcacctcctctatctgtccagcctcgcctcctgccaaatgaccctccctccgccaggtccactctgccctagctgcactctctccttcccccttttctttcttcttgtttctgaacagagagtgacatcaattacctgctaaagatggccctagagaaaatcgccttcttgccctttggctacctggtggaccagtggcgttggggggtcttcagtggacggaccccaccctctcgctacaacttcgactggtggtatcttcggtgagaagaggctcacccagggcacctcctgcctctaccagtcttgtcccttcctttctaacgtctgcccttttcccttcccactgaagcctcgccttcaggaagtcccctcacccccattttccaggctgtggaagggatgctgaggaaggggtgggataggggtggagccggtatcttttctgccagtctcaggcaacgtgtacagcctagggccttaggatttaaacatcctcttctagaaccaagtatcaggggatctgcccaccagttgcccggaatgaaacccattttgatgctggagccaagtttcacatcccaaacgtgacaccgtacatcaggtatctctgctctacccacatccctgctctgtcctccttcagacccaagggttgagagtcagaatgctccttcctgggctacgtacactccatttctgccttcttcccaacagacagcccttagtccagctgtcacctgcaggacaccggatagatagagcctgagcaaggggcctttgtctagctccccacgctcgttcctcctagcccccgtctgtgcccgcaggtacttcgtgagctttgtgctgcagttccagttccatcaagcactgtgcaaggaggcaggccaccagggcccactacaccagtgtgacatctaccagtccgcccaggcgggggccaagctcaagtatgtggcagggttccaaaacaaggggtaggggtcggagctgagggaagaggctggtggaggtcgtttctgcaggtagctgtctgctctggttgcaacaccacaaagccaggcacctgtggagtaactcttctctctttcctctactccttccatctcttcatccctccctccctctctttcttttctcccttcccttccttttcttccctccttccctctctccctctttctcctgtctctcttacatatcaaatatttattaagcaggccttcagctgccatgggccaggcaaatatcccttaccagcctcacagagacctcccccaccccccgcaaccctagagttcttttactagtgagggacaagtggacaatggtgctgttgtgggccccaccctgtgtcccctgtgcccacagtggtcactctgcttggcaggcaggtgttgcaggctggctgctccaggccctggcaggaggtactgaaggacctggtaggctcagatgccctggatgccaaggcactgctggagtacttccaaccggtcagccagtggctggaagagcagaatcagcggaatggcgaagtcctaggctggccagagaatcagtggcgtccaccgttacccgacaactatccagagggcattggtaaagctctgagtgagggtggactgggaccaagagaagtcctggcctctggcctctggcttctgggtcaaagcctcagcatcctggtcactttgctgccagctgagccccagtgtcctttgcttcagtgccaagccacccctgggctcatcctcagggccctaagcagaaatgggtatgtctttctctcagggtcctagagacagtgtgcccaagcctgagggcccttggggtcaggctggctggcacattgctctatgaggtcacactgcaggcttggctcttattggccggtgatgggagcttcagggctctgctttcctgcggccatgggccaaggttgggctactccaggactgcccagcttcctctttctgctgctctgctgtgggcaccatctgctggtcctgagtcaggtggccactgaccacgtgacagccaaccaggggataacaaaccaggcaacaaccagaagccagacaacaactcaccaagcaaccattgaccagacaacccagatcccaagtgggactcttggaagggggcagggccatgagggggcaaggagccaggggccagcaggagggaactccaatgagaccacaccctgtggtaaggaaggcaaggcctgtctgttttcctcctcacctcctgcagacctagagactgatgaagccaaggctgacaggttcgtggaagagtatgaccggacagcccaagtgttgttgaacgagtacgcagaggccaactggcaatataacaccaacattaccatagagggcagcaagatcctggtaggaaccacttccactaactccagcatgaaccatgtgcacacacacacacacacacacacacacacacacacacacagcaccaagctgtcaaaattggtgaaaagtctgaactgagtatttaaaagaatactcagtttccagtttagtgggcttggactctgcacctgagcaaatttctctcctttgttaccgttctctagttgaggattctcagaccgtaatccgttgtgcatcagtgacttataacattataacgttataacatcagtgttataaatgacttataaacgatgttagtgtcatccttaaaggactgacagcctgtttgtcagcactgagggatcattcccctgtaccttggccctttctttgtgacagagtatacattatactacagaaaaccaactagccatatgctccaaccactgggtgctaatgttgactgaagacccactatgtgctgcagtccctgggtccttggtacctcaacttctcttcctgcatacaccccttggcagctatgcataagcagtgaaattcttcccatttcaaagatgcaatcactgaatactcagggcaggcaggggtgggtgggtgtggtccttctaacagcacactgccttcatcctaatgacacctaaggcatgctaagctctttttccctacgtgctagactgcaggtgtctgccaaggaactcatactgtacctaatgtacagtatggtccaagttgaagtggagggtatttgtcagggcaaagataccagcctaatgaaacaacactcctcaccactacctttatagaccctcctacacggagaccataccaaatatcctgagcctgacactacctccttgtgccctcagcttgagaaaagcacggaggtatccaatcacaccctgaaatatggcacccgggccaagacatttgatgtgagcaactttcaaaactcttccatcaagcggatcc

SEQ ID NO: 4 - protein, 1312 aa, Mouse
MGAASGQRGRWPLSPPLLMLSLLVLLLQPSPAPALDPGLQPGNFSPDEAGAQLFAESYNSSAEVVMFQSTVASWAHDTNITEENARRQEEAALVSQEFAEVWGKKAKELYESIWQNFTDSKLRRIIGSIRTLGPANLPLAQRQQYNSLLSNMSRIYSTGKVCFPNKTATCWSLDPELTNILASSRSYAKLLFAWEGWHDAVGIPLKPLYQDFTAISNEAYRQDDFSDTGAFWRSWYESPSFEESLEHIYHQLEPLYLNLHAYVRRALHRRYGDKYVNLRGPIPAHLLGDMWAQSWENIYDMVVPFPDKPNLDVTSTMVQKGWNATHMFRVSEEFFTSLGLSPMPPEFWAESMLEKPTDGREVVCHASAWDFYNRKDFRIKQCTRVTMEQLATVHHEMGHVQYYLQYKDLHVSLRRGANPGFHEAIGDVLALSVSTPAHLHKIGLLDHVTNDIESDINYLLKMALEKIAFLPFGYLVDQWRWGVFSGRTPPSRYNFDWWYLRTKYQGICPPVARNETHFDAGAKFHIPNVTPYIRYFVSFVLQFQFHQALCKEAGHQGPLHQCDIYQSAQAGAKLKQVLQAGCSRPWQEVLKDLVGSDALDAKALLEYFQPVSQWLEEQNQRNGEVLGWPENQWRPPLPDNYPEGIDLETDEAKADRFVEEYDRTAQVLLNEYAEANWQYNTNITIEGSKILLEKSTEVSNHTLKYGTRAKTFDVSNFQNSSIKRIIKKLQNLDRAVLPPKELEEYNQILLDMETTYSLSNICYTNGTCMPLEPDLTNMMATSRKYEELLWAWKSWRDKVGRAILPFFPKYVEFSNKIAKLNGYTDAGDSWRSLYESDNLEQDLEKLYQELQPLYLNLHAYVRRSLHRHYGSEYINLDGPIPAHLLGNMWAQTWSNIYDLVAPFPSAPNIDATEAMIKQGWTPRRIFKEADNFFTSLGLLPVPPEFWNKSMLEKPTDGREVVCHPSAWDFYNGKDFRIKQCTSVNMEDLVIAHHEMGHIQYFMQYKDLPVTFREGANPGFHEAIGDIMALSVSTPKHLYSLNLLSTEGSGYEYDINFLMKMALDKIAFIPFSYLIDQWRWRVFDGSITKENYNQEWWSLRLKYQGLCPPVPRSQGDFDPGSKFHVPANVPYVRYFVSFIIQFQFHEALCRAAGHTGPLHKCDIYQSKEAGKLLADAMKLGYSKPWPEAMKLITGQPNMSASAMMNYFKPLTEWLVTENRRHGETLGWPEYNWAPNTARAEGSTAESNRVNFLGLYLEPQQARVGQWVLLFLGVALLVATVGLAHRLYNIRNHHSLRRPHRGPQFGSEVELRHS

SEQ ID NO: 5 - DNA, 21 bp, Artificial Sequence
  Oligonucleotide
ctagcttcctctgagagagcc

SEQ ID NO: 6 - DNA, 21 bp, Artificial Sequence
  Oligonucleotide
cctcggcactcgagttatagc

SEQ ID NO: 7 - DNA, 21 bp, Artificial Sequence
  Oligonucleotide
gcaggtcatggttatctggtg

SEQ ID NO: 8 - DNA, 21 bp, Artificial Sequence
  Oligonucleotide
ctgaggaagtgcaggacttac

(19) United States
(12) Patent Application Publication
(10) Pub. No.: US 2009/0217402 A1
(43) Pub. Date: Aug. 27, 2009

(54) METHODS AND COMPOSITIONS FOR MODULATING THE ACTIVITY OF PEPTIDASES IN MACROPHAGE AND MACROPHAGE-LIKE CELLS

(75) Inventors: Kenneth Bernstein, Atlanta, GA (US); Xiao Shen, Los Angeles, CA (US)

Correspondence Address: ALSTON & BIRD LLP, BANK OF AMERICA PLAZA, 101 SOUTH TRYON STREET, SUITE 4000, CHARLOTTE, NC, 28280-4000, US

(73) Assignee: Emory University, Atlanta, GA (US)

(21) Appl. No.: 11/914,881

(22) PCT Filed: May 18, 2006
(86) PCT No.: PCT/US06/19427

§ 371 Date: Jul. 8, 2008

Related U.S. Application Data
(60) Provisional application No. 60/683,457, filed on May 20, 2005.

(51) Int. Cl. A01K 67/00; C12N 15/00; C12N 5/00; A61K 35/12; A61K 39/00; C12N 15/63

(52) U.S. Cl. 800/18; 435/455; 435/325; 424/93.21; 424/184.1; 800/13; 435/320.1

(57) ABSTRACT

Methods and compositions are provided for modulating the activity of macrophage and macrophage-like cells, particularly by modulating the peptidase activity of such cells. In certain aspects, methods are provided for enhancing or suppressing the immune function of a host by increasing or decreasing, respectively, expression of a target peptidase in host macrophage and macrophage-like cells. Such methods for enhancing or suppressing the immune function of a host by increasing or inhibiting (suppressing or silencing) expression of a target peptidase in host macrophage and macrophage-like cells find use in methods for treating and/or preventing a number of diseases and conditions, including cancer, viral and/or bacterial infection, Alzheimer's disease, tissue transplant rejection, autoimmune diseases, and chronic inflammatory diseases. The invention also provides compositions and research tools related to these methods, including vectors for increasing or inhibiting the expression of a peptidase in macrophage and macrophage-like cells, macrophage and macrophage-like cells transfected with these vectors, genetically modified mice that over-express a target peptidase or in which expression of a target peptidase has been disrupted, and selective modulators of peptidase activity in macrophages as well as methods for identifying and using such modulators.

### FEDERALLY SPONSORED RESEARCH OR DEVELOPMENT

This invention was made with government support under grant numbers R01 DK051445-08 and R01DK055503-04 awarded by the National Institutes of Health. The United States government has certain rights in the invention.

### FIELD OF THE INVENTION

The invention relates to methods and compositions for modulating the activity of peptidases in macrophage and macrophage-like cells to modulate the immune response in a patient. In particular, the invention relates to methods for modulating the activity of peptidases such as angiotensin-converting enzyme (ACE) in macrophage and macrophage-like cells to modulate the immune response in a patient to treat cancer, infection, inflammation, autoimmune disorders, and tissue transplant rejection.

### BACKGROUND

Monocyte/macrophage and dendritic cells are key lines of cells providing natural immunity against infection, transplanted tissues and malignant tissues and play a central role in host defense through a variety of mechanisms involving both membrane-related and secretory events (Gordon et al. (1992) Curr. Opin, Immunol. 4:25-32; Fuller (1992) Brit. Med. Bull. 48:65-71). While macrophages and dendritic cells perform many different biological functions, it is a critical class of cells responsible for antigen presentation (immune sensitization) to other types of immune cells such as T-cells and B-cells. Macrophages perform this function by phagocytizing proteins which are catalytically cleaved into peptides. These peptides are then presented on the surface of macrophages for interaction with other cell types.

Macrophages can also become activated in a variety of pathologic circumstances. This process is associated with the maturation and activation of macrophages and the production of a variety of cytokines and reactive oxygen species. For example, macrophages also contribute to host defense through secretion of bacteriostatic and bactericidal proteins, cytokines and lipid mediators, as well as oxygen and nitrogen reactive intermediates. The secretory capacity of the macrophage is central to its function as these cells secrete over 100 distinct mediators and are located in every organ (Nathan (1987) J. Clin. Invest. 79:319-326).

The importance of macrophages in defense against microbes, immune surveillance, and destruction of tumor cells has been documented in man and in animal models characterized by the selective elimination of macrophages (Claassen et al. (1990) J. Immunol. Meth. 134:153-161). Aberrant activation of macrophage functions is associated with autoimmune disease as well as both chronic and acute inflammatory processes, while suppression of macrophage functions is associated with pathogenic infection, particularly recurrent infection for immunocompromised individuals such as burn patients, transplant patients, HIV infected individuals, cancer patients undergoing chemotherapy, and surgical patients. The ability to selectively modulate macrophage function and activity would therefore provide useful therapeutic approaches in the treatment of a variety of disorders and conditions associated with macrophage activity, including cancer, infection, inflammation, autoimmune disorders, and tissue transplant rejection.

### SUMMARY OF THE INVENTION

Aspects of the present disclosure include methods and compositions for modulating the activity of macrophage and macrophage-like cells. Certain aspects include methods and compositions for modulating the activity of macrophage and macrophage-like cells by modulating the peptidase activity of such cells. Aspects of the disclosure also include methods and compositions for enhancing or suppressing the immune function of a host by increasing or decreasing, respectively, peptidase expression and/or activity in host macrophage and macrophage-like cells. In additional aspects of the disclosure, methods and compositions for enhancing the immune function of a host by increasing peptidase expression in host macrophage and macrophage-like cells find use in methods for treating and/or preventing cancer and/or a tumor in a host, as well as methods for treating and/or preventing viral and/or bacterial infection in a host and methods for treating diseases in which increased peptidase activity has efficacy, such as Alzheimer's disease. In further aspects of the disclosure, methods and compositions for suppressing the immune function of a host by decreasing or inhibiting peptidase expression in host macrophage and macrophage-like cells find use in methods for treating and/or preventing rejection of tissue transplants, as well as methods for treating autoimmune diseases and methods for treating chronic inflammatory diseases.

Accordingly, certain aspects of the present disclosure relate to the modification of macrophage and macrophage-like cells to over-express or inhibit the expression of a biologically active peptidase (an enzyme that cleaves peptides). In an aspect of the disclosure, macrophage and/or macrophage-like cells are modified to over-express or inhibit the expression of the peptidase angiotensin converting enzyme (abbreviated as ACE). An aspect of modifying macrophage and macrophage-like cells to over-express or inhibit the expression of a peptidase includes modulating the transcription of a gene encoding a target peptidase in macrophage and macrophage-like cells. A certain aspect includes increasing the transcription of a gene encoding a peptidase in macrophages and macrophage-like cells, with a macrophage specific promoter, including but not limited to, the c-fms promoter. Other aspects include inhibition of peptidase expression by suppressing or silencing the expression of a peptidase in macrophage and macrophage-like cells, including but not limited to small interfering RNAs (siRNAs) or antisense nucleic acids. Other aspects of the disclosure include vectors for increasing or inhibiting the expression of a peptidase in macrophage and macrophage-like cells, wherein the vector includes a macrophage-specific promoter, such as c-fms promoter, or a sequence encoding siRNAs or antisense nucleic acids, where the vector also may include other polynucleotide sequences to aid in transfection and to aid in controlling transcription of a gene encoding the target peptidase. Macrophage and macrophage-like transfected with these vectors are also provided.

In another aspect of the disclosure, over-expression of a peptidase such as ACE modifies/augments macrophage function and is used to augment the natural immune defenses of a human or animal to treat primary or metastatic tumors. Another aspect of the disclosure relates to the over expression of a peptidase, including but not limited to, ACE in human macrophage and macrophage-like cells to modify the behavior of these cells to treat human cancer. In yet another aspect of the disclosure, over-expression of a peptidase such as ACE modifies/augments macrophage function and is used to augment the natural immune defenses of a human or animal to treat pathogenic (viral and/or bacterial) infection, particularly latent viral infections, including but not limited to HIV or hepatitis viruses. In another aspect over-expression of a peptidase, such as ACE, in macrophages is used as a treatment for Alzheimer's disease.

In another aspect of the disclosure, inhibition of expression of a peptidase such as ACE suppresses macrophage function and is used to suppress the natural immune defenses of a human or animal to treat or prevent rejection of tissue transplants. In another aspect of the invention, inhibition of expression of a peptidase such as ACE suppresses macrophage function and is used to suppress the natural immune defenses of a human or animal to treat autoimmune diseases, including but not limited to systemic lupus erythematosus. In yet another aspect of the invention, inhibition of expression of a peptidase such as ACE suppresses macrophage function and is used to suppress the natural immune defenses of a human or animal to treat chronic inflammatory diseases, including but not limited to rheumatoid arthritis.

Another aspect of this disclosure includes genetically modified mice that over express a peptidase, including but not limited to, ACE in macrophage and macrophage-like cells and methods for producing such genetically modified mice. Another aspect of this disclosure includes genetically modified knockout mice in which expression of a target peptidase has been disrupted, including but not limited to, ACE in macrophage and macrophage-like cells and methods for producing such genetically modified mice.

Still other aspects of this disclosure provide methods for identifying modulators of peptidase activity in macrophages. Other aspects of the disclosure provide methods for identifying modulators of peptidase activity which increase/augment or decrease/suppress the activity of peptidases in macrophage and macrophage-like cells but do not affect peptidase activity in other cells. Such modulators include, but are not limited to, compositions including polypeptides, antibodies, small molecules, a nucleic acid molecule or other compounds which up-regulate or down-regulate the activity of a peptidase in macrophage or macrophage-like cells. Such compounds may interact with cellular transcriptional elements and or the peptidase gene to increase or decrease translation of the peptidase or such compounds may directly interact with the peptidase to augment or suppress its activity. Methods of using such compounds for enhancing or suppressing the immune function of a host as well as for treating various diseases and conditions are also provided.

### BRIEF DESCRIPTION OF THE DRAWINGS

FIG. 1 shows results from testing of ACE activity in individual tissue extracts from wild-type (ACE wt/wt), heterozygous (ACE wt/10) and ACE 10/10 mice. Results are shown for plasma as well as lung, kidney, and spleen tissue.

FIG. 2 shows a comparison between the organization of the wild-type ACE gene (Top), a targeting construct for creation of ACE.10 mice (Middle), and the ACE.10 modified allele (Bottom). The targeting construct reflects placement of the c-fms promoter immediately 5′ of the ACE gene as part of the strategy for creating ACE.10 mice based on homologous recombination. For the wild-type ACE gene, the arrows represent the somatic and testis ACE promoters. Boxes show the 25 exons of ACE. The targeting construct contains a thymidine kinase gene to allow negative selection and a neomycin cassette for positive selection. Other details of the targeting vector are described in the Methods. In the modified ACE allele after targeting, the c-fins promoter directs ACE over-expression in macrophages and macrophage like cells.

FIG. 3 shows the results of Western blot analysis (using anti-ACE antibody) for ACE expression in the tissues of ACE 10/10 mice.

FIG. 4 shows FACS analysis of wild type and ACE 10/10 mice for ACE expression. In ACE 10/10 mice, ACE is expressed by macrophages, monocytes and, to a lesser extent, by neutrophils. ACE expression is not seen in T or B cells.

FIG. 5 provides photographs of tumor growth in littermate wild type mice (WT) and ACE 10/10 mice (KO) intracutaneously injected with 1×10⁶ B16-F10 melanoma cells. Large tumors grew in the WT mice while very small tumors were present in the KO mice. In panel C, a portion of skin was removed with the attached tumor, This shows the difference in growth between WT and KO mice.

FIG. 6 shows the difference in tumor size, measured in volume, present in littermate wild type (WT) and ACE 10/10 mice (KO) measured 11 and 14 days after the injection of 1×10⁶ B16-F10 melanoma cells.

FIG. 7 shows quantitation of tumor growth in wild type and ACE 10/10 mice measured 11 and 14 days after the injection of B 16-LS9 melanoma cells.

FIG. 8 shows quantitation of B16-F10 tumor growth in wild type and ACE 10/10 mice bred to a CD-1 (Swiss) outbred background.

FIG. 9 shows quantitation of B16-F10 tumor growth in wild type and ACE 10/10 mice with co-administration of an ACE inhibitor. Groups indicated by 10-I and WT-I were ACE 10/10 and wild type mice treated with the ACE inhibitor captopril.

FIG. 10 shows the treatment of wild type and ACE10/10 with the AT1 receptor antagonist losartan (indicated by A). This treatment did not inhibit the ability of the ACE 10/10 mice to resist melanoma growth.

FIG. 11, panel A, provides a photograph of a blood vessel from a B16-F10 tumor present in a wild type mouse. Very little inflammation was observed. In contrast, Panels B-D show photographs of blood vessels from a B16-F10 tumor present in an ACE 10/10 mouse. Far more inflammation was observed.

FIG. 12 shows tumor size from wild type mice lethally irradiated and then rescued by bone marrow transplantation from either ACE 10/10 or wild type mice followed by intradermal injection of B16-F10 melanoma cells. Mice receiving bone marrow from ACE 10/10 mice are more resistant to the tumor.

FIG. 13 shows ACE 10/10 mice injected with anti-CD4 and anti-CD8 monoclonal antisera (indicated by +), which eliminates T cells but does not eliminate monocytes or macrophages. Eliminating T cells eliminates the resistance of ACE 10/10 mice to tumor growth.

FIG. 14 shows TRP-2¹⁸⁰⁻¹⁸⁸ tetramer staining results from control and ACE 10/10 mice injected with a B 16 melanoma that was engineered to express ovalbumin. There was increased CD8 T cell tetramer binding in the ACE 10/10 mice.

FIG. 15 shows Ovalbumin²⁵⁷⁻²⁶⁴ tetramer staining results from control and ACE 10/10 mice injected with a B16 melanoma that was engineered to express ovalbumin. There was increased CD8 T cell tetramer binding in the ACE 10/10 mice.

FIG. 16 shows results from wild type mice injected intradermally with B16-F10 melanoma cells followed one week later by injection of the tumor nodules with 2 million wild type or ACE 10/10 macrophages. Injection of ACE 10/10 macrophages is more effective in reducing tumor size than macrophages from a wild-type mouse.

FIG. 17 shows T cell frequency in blood samples from wild-type (WT) and ACE 10/10 mice (KO) infected with Clone 13 at 8, 15, and 22 days following infection. After the initial phase of infection, ACE 10/10 mice showed a consistent increase in the blood T cell frequency for the GP33 peptide.

FIG. 18 shows Clone 13 serum blood viral titers from wild-type (WT) and ACE 10/10 mice (K(O) infected with Clone 13 at 8, 15, and 22 days following infection. ACE 10/10 mice cleared the Clone 13 virus from the blood dramatically faster than wild-type mice.

### DETAILED DESCRIPTION

The present invention provides methods and compositions for modulating the activity of macrophage and macrophage-like cells, particularly by modulating the peptidase activity of such cells. In certain methods, the invention relates to enhancing or suppressing the immune function of a host by increasing or decreasing, respectively, expression of a target peptidase in host macrophage and macrophage-like cells. As described in further detail herein, such methods for enhancing or suppressing the immune function of a host by increasing or inhibiting (suppressing or silencing) expression of a target peptidase in host macrophage and macrophage-like cells find use in methods for treating and/or preventing a number of diseases and conditions, including cancer, viral and/or bacterial infection, Alzheimer's disease, tissue transplant rejection, autoimmune diseases, and chronic inflammatory diseases. The invention also provides compositions and research tools related to these methods, including vectors for increasing or inhibiting the expression of a peptidase in macrophage and macrophage-like cells, macrophage and macrophage-like cells transfected with these vectors, genetically modified mice that over-express a target peptidase or in which expression of a target peptidase has been disrupted, and modulators of peptidase activity in macrophages as well as methods for identifying such modulators.

As used herein, the term “macrophage” and/or “macrophage-like cells” generally refers to macrophages, monocytes, and cells of macrophage/monocyte lineage including but not limited to dendritic cells, and any other similar cells which perform the functions generally associated with macrophages, such as antigen presentation to other classes of immune cells such as T-cells and B-cells in order to sensitize these cells to a particular target, including but not limited to viruses, bacterial cells, other foreign cells, cancer cells, and other undesired proliferating cells.

As used herein, the term “modulate,” “modify” and/or “modulator” generally refers to the act of promoting/activating or interfering with/inhibiting a specific function or behavior. For instance, a modulator of peptidase activity might activate peptidase function, or, alternatively, a modulator of peptidase activity might inhibit peptidase function. In both of these cases, the functional activity of a peptidase that is modified is the ability of the peptidase to cleave its target peptide. For example, modulation of angiotensin converting enzyme (ACE) refers to activation or inhibition of the ability of ACE to cleave angiotensin I and convert it into angiotensin II. A modulator may increase or decrease a certain activity or function relative to its natural state or relative to the average level of activity that would generally be expected. For example, an ACE modulator may activate or inhibit the ability of ACE to cleave angiotensin I and convert it into angiotensin II by at least 5%, at least 10%, at least 20%, at least 30%, at least 40%, at least 50%, at least 60%, at least 70%, at least 80%, at least 90%, and up to and including 100%, compared to its natural state or relative to the average level of activity that would generally be expected.

As used herein, the terms “enhance,” “increase,” and/or “augment” generally refer to the act of improving a function or behavior relative to the natural, expected or average. For instance, something that enhances or augments macrophage and macrophage-like cells function might improve the immune function of “enhanced” macrophage and macrophage-like cells by allowing these cells to recognize a greater number or a greater variety of diseased or disease carrying cells than un-enhanced macrophage and macrophage-like cells. Conversely, as used herein, the terms “inhibit,” “decrease,” and/or “suppress” generally refers to the act of disrupting, interfering, or worsening a function or behavior relative to the natural, expected or average. For instance, something that inhibits or suppresses macrophage and macrophage-like cell function might interfere with the immune function of “suppressed” macrophage and macrophage-like cells by interfering with the ability of such cells to recognize diseased or disease carrying cells compared to normal macrophage and macrophage-like cells.

As used herein, the term “host” or “organism” includes humans, mammals (e.g., cats, dogs, horses, etc.), living cells, and other living organisms. A living organism can be as simple as, for example, a single eukaryotic cell or as complex as a mammal.

### Target Peptidases

As described above, embodiments of the present invention relate to methods for modulating the activity of macrophage and macrophage-like cells by increasing or decreasing the expression of a target peptidase in such cells. For use within the methods and compositions of the present invention, the target peptidase includes but is not limited to angiotensin-converting enzyme (ACE). ACE is a zinc-peptidase responsible for the conversion of angiotensin I to the vasoconstrictor angiotensin II. As such, ACE plays a critical role in the renin-angiotensin system that is a central regulator of mammalian blood pressure. Pharmacologic agents that inhibit ACE are effective in reducing the elevated blood pressure accompanying hypertension.

While the role of angiotensin II in blood pressure control is well accepted, much scientific investigation has suggested a far broader physiologic role for angiotensin II. For example, Griffen et al. showed that chronic in vivo infusion of low dose angiotensin II led to a vascular hypertrophic response in blood vessels. (Griffin et al. (1991) “Angiotensin II Causes Vascular Hypertrophy in Part by a Non-Pressor Mechanism,” Hypertension, 17:626-635). They concluded that this was due, in part, to non-pressor mechanisms. Infusion of angiotensin II into animals injured with a vascular balloon catheter markedly exacerbated the resulting myoproliferative lesion. (Daemen et al. (1991), “Angiotensin II Induces Smooth Muscle Cell Proliferation in the Normal and Injured Rat Arterial Wall,” Circ. Res., 68:450-456). Conversely, ACE inhibitors have been shown to decrease neo-intimal formation after carotid injury. (Powell et al. (1989), “Inhibitors of Angiotensin-Converting Enzyme Prevent Myointimal Proliferation After Vascular Injury,” Science, 245:186-188). In fact, growth factor properties of angiotensin II have been demonstrated in fibroblasts, adrenal cortical cells, smooth muscle cells, cardiac myocytes, renal proximal tubular cells and tumor cells. (Timmermans et al (1993), “Angiotensin II Teceptors and Angiotensin II Receptor Antagonists,” Pharmacol. Reviews, 45:205-251).

In addition to the physiological effects described above for ACE and angiotensin II, angiotensin II was shown to induce the production of oxygen radicals, the release of cytokines such as TNF-β and the regulation of proliferation/apoptosis (Deshayes et al. (2005) Trends Endocrinol. Metab. 16:293-9). ACE also has many effects in addition to the production of angiotensin II. This is because ACE is a non-specific peptidase and can cleave many peptides apart from angiotensin I. While some of these non-angiotensin II peptides (such as bradykinin) play a role in blood pressure control, ACE also produces peptides that have immunologic effects. For example, Nakagawa et al. showed that ACE is important in the production of the immunodominant epitope of the HIV-1 protein gp160 (Nakagawa et al. (2000) Immunology 101:76-82). Other studies have also suggested a role of ACE in immunologic peptide presentation (Eisenlohr et al. (1992) Cell 71:963-972).

ACE is produced by a variety of tissue types including vascular endothelium, renal proximal tubular epithelium, areas of the gut and activated macrophages (somatic ACE). A unique isozyme of ACE is produced by developing male germ cells and is referred to as testis ACE. The production of ACE by vascular endothelium has several physiologic consequences. First, the production of angiotensin II by endothelial ACE in immediate proximity to vascular smooth muscle is thought to play a critical role in blood pressure control. However, ACE production in a normal blood vessel may be quite different from that during vascular injury due to hypertension or processes such as atherosclerosis. In these pathologies, vascular adventitial expression of ACE as well as the production of ACE by activated macrophages may generate local angiotensin II and exacerbate injury. (Weiss et al. (2001), “Angiotensin II and Atherosclerosis,” Am. J. Cardiol., 87:25C-32C; Yu, HT (2003) “Progression of Chronic Renal Failure,” Arch. Intern. Med., 163:1417-1429). Such considerations also apply to renal disease such as diabetes where there is glomerular accumulation of activated macrophages and the suggestion that macrophage produced angiotensin II mediates production of cytokines such as TGF-β. (Taal and Brenner, “Evolving Strategies for Renoprotection: Non-Diabetic Chronic Renal Disease,” Curr. Opin. Nephrol. Hypertens., 10:523-531). Indeed, both in animal models and in human disease, ACE inhibitors have been suggested as effective compounds for reducing a variety of proliferative pathologies including some of the deleterious effects of elevated lipids and diabetes (Anderson et al. (1985), “Control of Glomerular Hypertension Limits Glomerular Injury in Rats With Reduced Renal Mass,” J. Clin. Invest., 76:612-619).

ACE and the renin-angiotensin system have also been implicated in the response to neoplasia. Specifically, angiotensin II has been suggested as a pro-growth, pro-angiogenic agent. (Harrison et al. (2003), “Interactions of Angiotensin II with NAD(P)H Oxidase, Oxidant Stress and Cardiovascular Disease,” J. Renin Angiotensin Aldosterone Syst., 4:51-61; McFarlane et al. (2003) “Mechanisms by Which Angiotensin-Converting Enzyme Inhibitors Prevent Diabetes and Cardiovascular Disease,” Am. J. Cardiol., 91:30 H-37H; Ruiz-Ortega et al. (2001) “Proinflammatory Actions of Angiotensins,” Curr. Opin. Nephrol. Hypertens., 10:321-329). A variety of in vitro, animal model and human epidemiologic studies have investigated the effects of ACE inhibitors on cell growth or tumor progression. (Lindberg et al. (2004) “Angiotensin Converting Enzyme Inhibitors for Cancer Treatment?” Acta Oncol., 43:142-152; Molteni et al. (2003) “Cytostatic Properties of Some Angiotensin I Converting Enzyme Inhibitors and of Angiotensin II Type I Receptor Antagonists,” Curr. Pharm. Des., 9:751-761). Several of these studies have indicated an anti-proliferative response after ACE inhibitor administration.

As described above, for use within the methods and compositions of the present invention, a “target peptidase” includes but is not limited to ACE. Other target peptidases that may be used within the methods and compositions of the present invention include any peptidase expressed in macrophage and macrophage-like cells, in particular carboxypeptidases such as ACE. Carboxypeptidases that may be used within the methods and compositions of the present invention include ACE2 (a peptidase related to ACE; Donoghue et al. (2000) Circ. Res. 87:E1-9), carboxypeptidase A1, carboxypeptidase A2, carboxypeptidase A4, carboxypeptidase A6, carboxypeptidase B, carboxypeptidase D, carboxypeptidase N, carboxypeptidase E, mast cell carboxypeptidase, serine carboxypeptidases, metallocarboxypeptidases, Bleomycin hydrolase, N-acetylated-alpha-linked acidic dipeptidase II, and Xaa-Pro dipeptidase, as well as carboxypeptidases as described in Reznik and Fricker (2001) Cell Mol. Life. Sci. 58:1790-1804; Skidgel and Erdos (1998) Immunol. Rev. 161:129-141; Skidgel (1988) Trends Pharmacol. Sci. 9:299-304; Goldstein et al. (1989) Prog. Clin. Biol. Res. 297:145-154; Remington (1993) Curr. Opin. Biotechnol. 4:462-468; Vendrell et al. (2000) Biochim. Biophys. Acta 1477:284-298; all of which are hereby incorporated by reference. These references also provide methods for identifying peptidases that are expressed in macrophage and macrophage-like cells, and that therefore may be useful as a target peptidase within the methods and compositions of the present invention, in particular carboxypeptidases.

Also for use within the methods and compositions of the present invention, are variants of such target peptidases, including but not limited to, variants of ACE. The term “variant” as used herein refers to modified amino acid sequences derived from that of a target peptidase, particularly ACE (SEQ ID NO: 1). Generally, such variants for use in the methods of the invention will have at least about 40%, 45%, 50%, 55%, 60%, 65%, 70%, 75%, 80%, 85%, 90%, 91%, 92%, 93%, 94%, 95%, 96%, 97%, 98%, 99% or more sequence identity to amino acid sequences derived from that of target peptidases, particularly ACE (SEQ ID NO:1), as determined by sequence alignment programs and parameters described elsewhere herein. Such biologically active variants for use in the methods of the invention may differ from ACE by as few as 1-15 amino acid residues, as few as 1-10, such as 6-10, as few as 5, as few as 4, 3, 2, or even 1 amino acid residue.

The terms “percent sequence identity” or “percent sequence similarity” as used herein refer to the degree of sequence identity between two sequences as determined using the algorithm of Karlin and Altschul (1990) Proc. Natl. Acad. Sci. USA 87: 2264, modified as in Karlin and Altschul (1993) Proc. Natl. Acad. Sci. USA 90: 5873-5877. Such an algorithm is incorporated into the NBLAST and XBLAST programs of Altschul et al. (1990) J. Mol. Biol. 215: 403-410. BLAST protein searches are performed with the XBLAST program, score=50, wordlength=3, to obtain amino acid sequences homologous to a reference polypeptide. To obtain gapped alignments for comparison purposes, Gapped BLAST is utilized as described in Altschul et al. (1997) Nucleic Acids Res. 25: 3389-3402. When utilizing BLAST and Gapped BLAST programs, the default parameters of the respective programs (e.g. XBLAST and NBLAST) are used (See http://www.ncbi.nlm.nih.gov). Other algorithms, equivalent programs, and default settings may also be suitable. By “equivalent program” is intended any sequence comparison program that, for any two sequences in question, generates an alignment having identical amino acid residue matches and an identical percent sequence identity when compared to the corresponding alignment generated by GAP Version 10.

The variant peptidases used in the methods of the present invention are biologically active, that is they possess the desired biological activity of cleaving a target peptide as described elsewhere herein (for example, a variant ACE that possesses the desired biological activity of cleaving angiotensin I and converting it into angiotensin II). Assays for determining the ability of a peptidase to cleave its target peptide are well-known in the art (see, e.g., for ACE, Skeggs et al. (1954) J. Exp. Med. 99:274-282; Cushman & Cheung (1971) Biochem. Pharmacol. 20:1637-1648; Ehlers et al. (1986) Biochim. Biophys. Acta 883:361-372; and Hooper (1990) Biochem. J. 270:840-841).

In another embodiment of the present invention, the methods involve the use of fragments of any of the variant peptidases described herein, for example ACE, so long as such fragments are biologically active as described elsewhere herein. By “fragment” is intended a portion of the amino acid sequence, and generally comprise at least 15, 25, 30, 50, 100, 150, 200, or 250 contiguous amino acids, or up to the total number of amino acids present in a variant paptidase described herein, for example ACE.

### Modulating Peptidase Expression in Macrophage and Macrophage-like Cells

As described above, embodiments of the present invention relate to methods for modulating the activity of macrophage and macrophage-like cells by increasing or decreasing the expression of a target peptidase in such cells. Such methods may employ a nucleotide construct that is capable of directing the expression of at least one polypeptide, or the transcription of at least one RNA, such as, for example, an antisense RNA that is complementary to at least a portion of an mRNA. Typically such a nucleotide construct is comprised of a coding sequence for a protein or an RNA operably linked to 5′ and 3′ transcriptional regulatory regions. In addition, it is recognized that methods of the present invention do not depend on the incorporation of the entire nucleotide construct into the genome, only that the cell, tissue, or patient is altered as a result of the introduction of the nucleotide construct into a cell.

The use of the term “nucleotide constructs” herein is not intended to limit the present invention to nucleotide constructs comprising DNA. Those of ordinary skill in the art will recognize that nucleotide constructs, particularly polynucleotides and oligonucleotides, comprised of ribonucleotides and combinations of ribonucleotides and deoxyribonucleotides may also be employed in the methods disclosed herein. Thus, the nucleotide constructs of the present invention encompass all nucleotide constructs that can be employed in the methods of the present invention including, but not limited to, those comprised of deoxyribonucleotides, ribonucleotides, and combinations thereof. Such deoxyribonucleotides and ribonucleotides include both naturally occurring molecules and synthetic analogues. The nucleotide constructs of the invention also encompass all forms of nucleotide constructs including, but not limited to, single-stranded forms, double-stranded forms, hairpins, stem-and-loop structures, and the like.

As used herein, the term “polynucleotide” generally refers to any polyribonucleotide or polydeoxyribonucleotide, which may be unmodified RNA or DNA or modified RNA or DNA. Thus, for instance, polynucleotides as used herein refers to, among others, single- and double-stranded DNA, DNA that is a mixture of single- and double-stranded regions, single- and double-stranded RNA, and RNA that is mixture of single- and double-stranded regions, hybrid molecules comprising DNA and RNA that may be single-stranded or, more typically, double-stranded or a mixture of single- and double-stranded regions. The terms “nucleic acid,” “nucleic acid sequence,” or “oligonucleotide” also encompasses a polynucleotide as defined above.

As used herein, the term polynucleotide includes DNAs or RNAs as described above that contain one or more modified bases. Thus, DNAs or RNAs with backbones modified for stability or for other reasons are “polynucleotides” as that term is intended herein. Moreover, DNAs or RNAs comprising unusual bases, such as inosine, or modified bases, such as tritylated bases, to name just two examples, are polynucleotides as the term is used herein.

It will be appreciated that a great variety of modifications have been made to DNA and RNA that serve many useful purposes known to those of skill in the art. The term polynucleotide as it is employed herein embraces such chemically, enzymatically or metabolically modified forms of polynucleotides, as well as the chemical forms of DNA and RNA characteristic of viruses and cells, including simple and complex cells, inter alia.

By way of example, a polynucleotide sequence of the present disclosure may be identical to the reference sequence, that is be 100% identical, or it may include up to a certain integer number of nucleotide alterations as compared to the reference sequence. Such alterations are selected from the group including at least one nucleotide deletion, substitution, including transition and transversion, or insertion, and wherein said alterations may occur at the 5′ or 3′ terminal positions of the reference nucleotide sequence or anywhere between those terminal positions, interspersed either individually among the nucleotides in the reference sequence or in one or more contiguous groups within the reference sequence. The number of nucleotide alterations is determined by multiplying the total number of nucleotides in the reference nucleotide by the numerical percent of the respective percent identity (divided by 100) and subtracting that product from said total number of nucleotides in the reference nucleotide. Alterations of a polynucleotide sequence encoding the polypeptide may alter the polypeptide encoded by the polynucleotide following such alterations.

The term “codon” means a specific triplet of mononucleotides in the DNA chain. Codons correspond to specific amino acids (as defined by the transfer RNAs) or to start and stop of translation by the ribosome.

The term “degenerate nucleotide sequence” denotes a sequence of nucleotides that includes one or more degenerate codons (as compared to a reference polynucleotide molecule that encodes a polypeptide). Degenerate codons contain different triplets of nucleotides, but encode the same amino acid residue (e.g., GAU and GAC triplets each encode Asp).

“Operably linked” refers to a juxtaposition wherein the components are configured so as to perform their usual function. For example, control sequences or promoters operably linked to a coding sequence are capable of effecting the expression of the coding sequence, and an organelle localization sequence operably linked to protein will direct the linked protein to be localized at the specific organelle.

As used herein, the term “exogenous DNA” or “exogenous nucleic acid sequence” or “exogenous polynucleotide” refers to a nucleic acid sequence that was introduced into a cell or organelle from an external source. Typically the introduced exogenous sequence is a recombinant sequence.

As used herein, the term “transfection” refers to the introduction of a nucleic acid sequence into the interior of a membrane enclosed space of a living cell, including introduction of the nucleic acid sequence into the cytosol of a cell as well as the interior space of a mitochondria, nucleus or chloroplast. Numerous methods of gene delivery may be implemented within the methods of the present invention. These include, but are not limited to, chemical methods such as calcium phosphate, physical methods such as electroporation, and viral gene transfer techniques (reference: Methods in Molecular Biology: Gene Delivery to Mammalian Cells, Humana Press 2004, Volumes 245 and 246, edited by William C. Heiser). The nucleic acid may be in the form of naked DNA or RNA, associated with various proteins or the nucleic acid may be incorporated into a vector.

As used herein, the term “vector” or “expression vector” is used in reference to a vehicle used to introduce a nucleic acid sequence into a cell. A vector may include a DNA molecule, linear or circular, which includes a segment encoding a polypeptide of interest operably linked to additional segments that provide for its transcription and translation upon introduction into a host cell or host cell organelles. Such additional segments may include promoter and terminator sequences, and may also include one or more origins of replication, one or more selectable markers, an enhancer, a polyadenylation signal, etc. Expression vectors are generally derived from yeast or bacterial genomic or plasmid DNA, or viral DNA, or may contain elements of both.

As used herein, the term “organelle” refers to cellular membrane bound structures such as the chloroplast, mitochondrion, and nucleus. The term “organelle” includes natural and synthetic organelles.

In certain embodiments of this disclosure, macrophage and/or macrophage-like cells are modified to over-express a target peptidase, such as ACE. It is contemplated that the target nucleic acids of the disclosure may be “overexpressed”, i.e., expressed in increased levels relative to its natural expression in human cells, or even relative to the expression of other proteins in the recombinant host cell. Such overexpression may be assessed by a variety of methods for assessing the expression level of target peptidases, including radio-labeling and/or protein purification. However, simple and direct methods are preferred, for example, those involving SDS/PAGE and protein staining or western blotting, followed by quantitative analyses, such as densitometric scanning of the resultant gel or blot. A specific increase in the level of the target protein or peptide in comparison to the level in natural human cells is indicative of overexpression, as is a relative abundance of the specific protein in relation to the other proteins produced by the host cell and, e.g., visible on a gel.

Over-expression of a target peptidase may be achieved through the use of a macrophage-specific promoter. As used herein, the term “macrophage-specific promoter” and/or “promoter which is selectively activated in macrophage and macrophage-like cells” refers generally to a promoter and/or promoter construct which is designed to allow regulation of transcription of a downstream nucleic acid sequence so that transcription may be activated in macrophage or macrophage-like cells without necessarily activating transcription in other cells. In an embodiment of the disclosure, the promoter is an exogenous polynucleotide that is introduced into the host cell to control transcription of a gene encoding a target peptidase, such as ACE. In one embodiment of the disclosure, the promoter is a c-fms promoter. The c-fms gene encodes a receptor for macrophage colony-stimulating factor-1 and its expression is largely restricted to macrophage lineage cells and placental trophoblast (Lichanska et al. (1999) “Differentiation of the Mononuclear Phagocyte System During Mouse Embryogenesis: The Role of Transcription Factor PU.1,” Blood, 94:127-138), thus the use of the c-fms promoter to control the expression of a target peptidase allows the over-expression of the peptidase to be specific to macrophage and macrophage-like cells. The exogenous polynucleotide (e.g., the c-fms promoter) can be obtained from various sources, preferably from the host organism.

Certain embodiments of this disclosure relate to expression vectors including a macrophage-specific promoter, such as the c-fms promoter, along with various regulatory elements, to be used for introducing the macrophage-specific promoter into a cell to control expression of a target peptidase. Such a vector can be made according to methods well known to skill in the art of recombinant expression, and may contain various additional nucleotide sequences, in addition to the promoter sequence, such as replicon, reporter and control sequences.

Many standard techniques are available to construct expression vectors containing the appropriate nucleic acids and transcriptional/translational control sequences in order to achieve protein or peptide expression in a variety of host-expression systems. Cell types available for expression include, but are not limited to, bacteria, such as E. coli and B. subtilis transformed with recombinant phage DNA, plasmid DNA or cosmid DNA expression vectors. In general, plasmid vectors containing replicon and control sequences that are derived from species compatible with the host cell are used in connection with these hosts. The vector ordinarily carries a replication site, as well as marking sequences that are capable of providing phenotypic selection in transformed cells.

Specific initiation signals may also be required for efficient translation of the claimed isolated nucleic acid coding sequences. These signals include the ATG initiation codon and adjacent sequences. Exogenous translational control signals, including the ATG initiation codon, may additionally need to be provided. One of ordinary skill in the art would readily be capable of determining this need and providing the necessary signals. It is well known that the initiation codon must be in-frame (or in-phase) with the reading frame of the desired coding sequence to ensure translation of the entire insert. These exogenous translational control signals and initiation codons can be of a variety of origins, both natural and synthetic. The efficiency of expression may be enhanced by the inclusion of appropriate transcription enhancer elements or transcription terminators.

The present disclosure also includes recombinant cells into which an exogenous polynucleotide sequence encoding for a macrophage-specific promoter has been introduced. As used herein, the terms “engineered” and “recombinant” cells are intended to refer to a cell into which an exogenous DNA segment or gene, such as a cDNA or gene has been introduced. Recombinant cells include those having an introduced cDNA or genomic DNA, and also include genes positioned adjacent to a promoter not naturally associated with the particular introduced gene. In embodiments of recombinant cells according to the present disclosure, the exogenous polynucleotide sequence encoding for a macrophage-specific promoter has been introduced such that a target peptidase is under the control of the macrophage specific promoter, thus allowing for the over-expression of the target peptidase. To bring a coding sequence “under the control of” a promoter, one positions the promoter generally about 1 to 50 nucleotides “upstream” of the 5′ end of the translational initiation site of the reading frame of the target gene. The inserted promoter stimulates transcription of the “downstream” DNA and promotes expression of the encoded protein. This is the meaning of “recombinant expression” in the context used here. In embodiments of this disclosure, the gene encoding for the target peptidase may be located in the host cell genome and/or it may be encoded by an exogenous polynucleotide sequence and introduced to the host cell in a vector in conjunction with the promoter sequence.

For long-term, high-yield production of target proteins, stable expression is preferred. For example, cell lines that stably express constructs encoding proteins may be engineered. Rather than using expression vectors that contain viral origins of replication, host cells can be transformed with vectors controlled by appropriate expression control elements (e.g., promoter, enhancer, sequences, transcription terminators, polyadenylation sites, etc.), and a selectable marker. Following the introduction of foreign DNA, engineered cells may be allowed to grow for 1-2 days in an enriched medium, and then are switched to a selective medium. The selectable marker in the recombinant plasmid confers resistance to the selection and allows cells to stably integrate the plasmid into their chromosomes and grow to form foci, which in turn can be cloned and expanded into cell lines.

A number of selection systems may be used, including, but not limited to, the herpes simplex virus thymidine kinase, hypoxanthine-guanine phosphoribosyltransferase and adenine phosphoribosyltransferase genes, in tk⁻, hgprt⁻ or aprt⁻ cells, respectively. Also, antimetabolite resistance can be used as the basis of selection for dhfr, which confers resistance to methotrexate; gpt, which confers resistance to mycophenolic acid; neo, which confers resistance to the aminoglycoside G-418; and hygro, which confers resistance to hygromycin.

In one embodiment of the disclosure the vector containing the exogenous polynucleotide sequence encoding for the macrophage-specific promoter, such as the c-fms promoter, is engineered to allow for direct insertion of the promoter sequence into the host cell genome upstream of the polynucleotide sequence encoding for a target peptidase, such as ACE.

In other embodiments of the present invention, expression of a target peptidase, such as ACE, is inhibited (suppressed or silenced) in host macrophage and macrophage-like cells (i.e., expressed in decreased levels relative to its natural expression in human cells, or even relative to the expression of other proteins in the recombinant host cell). Such inhibited expression may be assessed by a variety of methods for assessing the expression level of target peptidases, as described elsewhere herein.

Methods for suppressing or silencing the expression of a peptidase in macrophage and macrophage-like cells are well known in the art, and include the use of small interfering RNAs (siRNAs) or antisense nucleic acids. In mammalian cells, short, e.g., 21 nt, double stranded siRNA's have been shown to be effective at inducing an RNA interference (RNAi) response, which is a mechanism to suppress gene expression in a sequence specific manner (See, e.g., Elbashir et al. (2001) Nature 411:494-498; Sharp (1999) Genes Dev. 13:139-141; and Cathew (2001) Curr. Op. Cell Biol. 13:244-248). This mechanism may be used to down-regulate expression levels of target genes. Antisense polynucleotides are nucleic acids complementary to, and which can preferably hybridize specifically to, a coding mRNA nucleic acid sequence for a target peptidase or a subsequence thereof. Binding of the antisense polynucleotide to the mRNA reduces the translation and/or stability of the mRNA. In the context of this invention, antisense polynucleotides can comprise naturally-occurring nucleotides, or synthetic species formed from naturally-occurring subunits or their close homologs. Such antisense polynucleotides can readily be synthesized using recombinant means, or can be synthesized in vitro, and the ability to derive an antisense oligonucleotide based upon a cDNA sequence encoding a given peptidase is well known in the art (See, e.g., Stein and Cohen (1988) Cancer Res. 48:2659-2668; and van der Krol et al. (1988) BioTechniques 6:958-976).

Accordingly, in one embodiment of the invention, a method for inhibiting the activity of a target peptidase in a macrophage or macrophage-like cell is provided comprising introducing into the cell a nucleotide construct comprising a polynucleotide sequence operably linked to at least one promoter that is functional in the cell, where the polynucleotide comprises a nucleotide sequence encoding a modulator of peptidase expression in macrophage and macrophage-like cells, where the modulator inhibits transcription of a nucleotide sequence encoding the target peptidase. Such modulators that inhibit transcription of a nucleotide sequence encoding a target peptidase include siRNAs and antisense polynucleotides, as described above.

### Methods for Regulating Immune Function and Treating Disease

Another aspect of this disclosure relates to methods and compositions for modulating the expression of a peptidase such as ACE (i.e., over-expression or inhibition of expression) in macrophage and macrophage-like cells to modulate (up-regulate or suppress) immune function and as an approach to the treatment of various diseases and conditions, including cancer, viral and/or bacterial infection, Alzheimer's disease, tissue transplant rejection, autoimmune diseases, and chronic inflammatory diseases. As used herein, the term “treating” includes alleviating the symptoms associated with a specific disorder or condition and/or preventing or eliminating said symptoms.

Such methods of treatment involve the isolation of macrophage and macrophage-like cells from a patient, modulation of expression of a peptidase such as ACE (i.e., over-expression or inhibition of expression) in these macrophages as described elsewhere herein, and re-introduction of these modified cells into the patient. Using standard methods of cell preparation, it is straightforward to isolate human monocytes and macrophages. (Zareie et al. (2001) “Monocyte/Macrophage Activation by Normal Bacteria and Bacterial Products: Implications for Altered Epithelial Function in Crohn's Disease,” Am. J. Pathol., 158:1101-1109). Using published methods, these macrophages can be proliferated in vitro to obtain large numbers of cells. (Davies and Gordon (2005) “Isolation and Culture of Human Macrophages,” Methods Mol. Biol., 290:105-116). A peptidase such as ACE can be over expressed in these cells using published methods of transfection (e.g. virus mediated transfection) in conjunction with the human c-fms promoter as the mediator of protein over-expression. (Partridge and Oreffo (2004) “Gene Delivery in Bone Tissue Engineering Progress and Prospects Using Viral and Nonviral Strategies,” Tissue Eng., 10:295-307). The modified cells can then be re-injected back into the cancer patient from which they originated.

In one embodiment, we believe this will be a novel and useful approach to the treatment of human cancers including human metastatic and micrometastatic tumors including, but not limited to, melanoma or micrometastases of melanoma. Thus, embodiments of this disclosure include methods and compositions for treating cancer in a patient by increasing peptidase expression in the patient's macrophage and macrophage-like cells. Such a strategy could be used as a prophylactic to prevent cancer or as a therapeutic after surgery, either alone or in combination with chemotherapy, to prevent tumor re-growth.

Cancer is the second leading cause of death among Americans. According to the American Cancer Society, approximately 1.3 million Americans are estimated to be diagnosed with invasive cancer in 2003. The National Cancer Institutes estimates that approximately 8.9 million Americans had a history of cancer in 2003, and approximate 1,500 cancer-related deaths per day are expected in 2003. Because of the staggering number of cancer-related deaths and new cases, new medicines and methods of treatment are needed. Although recent advances have increased our understanding of some of the mechanisms leading to cancer, effective treatments for cancer remain in high demand.

Cancer can be defined as an abnormal growth of tissue characterized by a loss of cellular differentiation. This term encompasses a large group of diseases in which there is an invasive spread of undifferentiated cells from a primary site to other parts of the body where further undifferentiated cellular replication occurs, which eventually interferes with the normal functioning of tissues and organs.

Each cancer is characterized by the site, nature, and clinical cause of undifferentiated cellular proliferation. The underlying mechanism for the initiation of cancer is not completely understood; however, about 80% of cancers may be triggered by external stimuli such as exposure to certain chemicals, tobacco smoke, ultra violet rays, ionizing radiation, and viruses. Development of cancer in immunosuppressed individuals indicates that the immune system is an important factor controlling the replication and spread of cancerous cells throughout the body.

Current treatments of cancer and related diseases have limited effectiveness and numerous serious unintended side effects. Cancer therapy is currently divided into many categories including surgery, radiation therapy, chemotherapy, bone marrow transplantation, stem cell transplantation, hormonal therapy, immunotherapy, antiangiogenic therapy, targeted therapy and gene therapy and others. These treatments have largely progressed incrementally during more than thirty years of intensive research to discover the origins of cancer and devise improved therapies for cancer and related diseases.

The principal way of treating cancer is through toxic chemotherapeutic agents. Current research strategies emphasize the search for effective therapeutic modes with less risk, including the use of natural products and biological agents. We have long desired a way of augmenting natural immune responses against cancer and cancer metastases.

To our knowledge, no one has ever discovered or even postulated that the over expression of a peptidase such as ACE within macrophages would modify the macrophage response to a tumor such as the B16-F10 melanoma. Whatever the precise mechanism, modification of macrophage function by increased peptidase expression is an approach to cancer treatment complimentary to other approaches to malignancy control. For example, as described in detail in the Experimental section below, the results observed in the ACE 10/10 mice involved no chemotherapeutic agents or extraneous administration of adjuvants. Thus, what was discovered is that there is a relatively simple and nontoxic ways to dramatically change the activity of macrophage function in a host, and that this presents new and useful methods for preventing the growth and/or spread of cancer and/or a tumor including, but not limited to, melanoma. Thus, another aspect of this disclosure relates to a new mechanism to augment cancer therapy in humans. For instance the methods and compositions of this disclosure could be used to treat a patient with cancer in conjunction with more traditional cancer treatments, including but not limited to chemotherapy, radiation, and surgical procedures.

In another embodiment the up-regulation of the immune response provided by the over-expression of peptidases in macrophage and macrophage-like cells augments the response to pathogenic infection in addition to malignancy. Thus, another embodiment of the disclosure includes providing a treatment for bacterial and/or viral infections (e.g., latent viral infections including, but not limited to, HIV) in a host by increasing the peptidase activity of host macrophages. These methods are particularly useful in the treatment of recurrent infection for immunocompromised individuals such as burn patients, transplant patients, HIV infected individuals, cancer patients undergoing chemotherapy, and surgical patients.

Bacterial infections which may be treated within the methods of the present invention for up-regulation of the immune response provided by the over-expression of peptidases such as ACE in macrophage and macrophage-like cells include those caused by members of the following genera and species: Agrobacterium tumefaciens, Aquaspirillum, Bacillus, Bacteroides, Bordetella pertussis, Borrelia burgdorferi, Brucella, Burkholderia, Campylobacter, Chliamydia, Clostridium, Corynebacterium diptheriae, Coxiella burnetii, Deinococcus radiodurans, Enterococcus, Escherichia, Francisella tularemsis, Geobacillus, Haemophilus influenzae, Helicobacter pylori, Lactobacillus, Listeria monocytogenes, Mycobacterium, Mycoplasma, Neisseria meningitidis, Pseudomonas, Rickettsia, Salmonella, Shigella, Staphylococcus, Streptococcus, Streptomyces coelicolor, Vibro, and Yersinia. In a preferred embodiment, such infections include those caused by Escherichia coli, Helicobacter pylori, Listeria monocytogenes, Salmonella typhimurium, Shigella flexneri, and Mycobacterium tuberculosis (TB).

Viral infections which may be treated within the methods of the present invention for up-regulation of the immune response provided by the over-expression of peptidases such as ACE in macrophage and macrophage-like cells include those caused by members of the following virus families: Adenoviridae, Areiiaviridae, Astroviridae, Bacteriophages, Baculoviridae, Bunyaviridae, Calciviridae; Coronaviridae, Deltavirus, Filoviridae, Flaviviridae, Gem iniviridae, Hepadnaviridae, Herpesviridae, Nodaviridae, Orthomyxoviridae, Papovaviridae, Paramyxoviridae, Parvoviridae, Plzycodnzaviridae, Picornaviridae, Poxyiridae, Reoviridae, Retroviridae, Rhabdoviridae, Tobamoviridae, and Toqaviridae. In a preferred embodiment, such infections include those caused by human immunodeficiency viruses (e.g., HIV-1), Herpesviridae viruses (e.g., herpes simplex viruses 1 and 2, varicella zoster virus, Epstein-Barr virus, and cytomegalovirus), and hepatitis viruses (e.g., Hepatitis A, B, C, D, E, and G).

Additionally, elevated levels of ACE expression in macrophage and macrophage-like cells may be associated with reducing the progression of Alzheimer's Disease. It is believed that a peptidase, such as ACE, may act to reduce plaque size and prevent formation of new plaques associated with Alzheimer's Disease. Thus, another embodiment of the disclosure includes increasing the peptidase expression in macrophages to provide a treatment for Alzheimer's Disease.

In another aspect of the present invention, inhibition of activity and/or expression of a peptidase such as ACE in macrophage and macrophage-like cells is used to suppress the natural immune defenses of a human or animal to treat or prevent various diseases and conditions associated with aberrant or undesirable activation of the immune response, including rejection of tissue transplants, autoimmune diseases, and chronic inflammatory diseases.

Autoimmune diseases which may be treated within the methods of the present invention for suppression of the immune response provided by the inhibition of activity and/or expression of a peptidases such as ACE in macrophage and macrophage-like cells include, but are not limited to, systemic lupus erythematosus, Sjögren's syndrome, and rheumatoid arthritis (which may also be characterized as a chronic inflammatory disease).

Chronic inflammatory diseases which may be treated within the methods of the present invention for suppression of the immune response provided by the inhibition of activity and/or expression of a peptidase such as ACE in macrophage and macrophage-like cells include, but are not limited to, rheumatoid arthritis (which may also be characterized as an autoimmune disease), osteoarthritis, inflammatory lung disease, inflammatory bowel disease, atherosclerosis, and psoriasis.

As described elsewhere herein, compounds or agents that inhibit the activity and/or expression of a peptidase such as ACE in macrophage and macrophage-like cells include, but are not limited to antibodies, small molecules, a nucleic acid molecule or other compounds which inhibit the activity of a peptidase in macrophage or macrophage-like cell (see, e.g., ACE inhibitors as described in Dendorfer et al. (2005) Handb. Exp. Pharmacol. 170:407-442; Fyhrquist (1986) Drugs 5:33-39; Nelson et al. (1986) Am. J. Med. 81:13-18; Ondetti (1988) Circulation 77:14-13; Belz et al. (1988) Clin. Pharmacokinet. 15:295-318; Kostis (1989) J. Hum. Hypertens. 3:119-125; Thind (1990) Cardiovasc Drugs Ther. 4:199-206; and Hooper (1991) Int. J. Biochem. 23:641-647). Such compounds or agents may interact with cellular transcriptional elements and or the peptidase gene to inhibit translation of the peptidase or such compounds may directly interact with the peptidase to suppress its activity. In a particular embodiment, such compounds or agents inhibit peptidase expression and/or activity in macrophage and macrophage-like cells but do not inhibit peptidase expression and/or activity in other host cells. Such agents are administered in a therapeutically effective amount to treat the desired disease or condition as described above. By “therapeutically effective amount” is intended an amount of a compound or agent that inhibits the activity and/or expression of a peptidase as defined elsewhere herein (i.e., the ability of the peptidase to cleave its target peptide, for example inhibiting the ability of ACE to cleave angiotensin I and convert it into angiotensin II).

### Cell Lines and Transgenic Animals

Another embodiment of the invention includes a cell line or transgenic animal which has been engineered to either over-express at least one target peptidase or in which expression of at least one target peptidase has been disrupted in macrophage and macrophage-like cells. As used herein, a transgenic cell line or transgenic animal can include a host cell having exogenous polynucleotide incorporated therein. In this regard, transgenic animals comprise exogenous DNA incorporated into the animal's cells to effect a permanent or transient genetic change, preferably a permanent genetic change. The exogenous nucleic acid sequence may be present as an extrachromosomal element or stably integrated in all or a portion of the animal's cells, especially in germ cells. Permanent genetic change is generally achieved by introduction of the DNA into the genome of the cell. Vectors for stable integration include plasmids, retroviruses and other animal viruses, YACS, and the like. Generally, transgenic animals are mammals, most typically mice. Such transgenic animals include, but are not limited to, mammals such as mice, rats, guinea pigs, dogs, cats, pigs, cows, goats, sheep, and non-human primates.

A particular embodiment of this disclosure includes genetically modified mice that over-express a peptidase such as ACE in macrophage and macrophage-like cells and methods for producing such genetically modified mice. In another particular embodiment of this disclosure, genetically modified mice in which expression of at least one target peptidase such as ACE has been disrupted in macrophage and macrophage-like cells are provided (e.g., knock-out mice in which one or more genes encoding a target peptidase such as ACE have been made inoperable).

In one embodiment, a transgenic animal is provided comprising at least one macrophage or macrophage-like cell having stably incorporated into its genome a polynucleotide sequence operably linked to at least one promoter that is functional in the cell, wherein the polynucleotide comprises a nucleotide sequence encoding a target peptidase, wherein at least one of the promoters increases transcription of the nucleotide sequence encoding the target peptidase, and wherein the target peptidase is over-expressed in the macrophage or macrophage-like cell. In other aspects, the target peptidase over-expressed in the macrophage or macrophage-like cell is a carboxypeptidase, particularly where the carboxypeptidase is an angiotensin-converting enzyme or biologically active fragment thereof. In particular aspects, the transgenic animal is a mouse, and peptidase expression is directed by a c-fms promoter sequence comprising the sequence set forth in SEQ ID NO:2. In other aspects, the nucleotide sequence encoding the target peptidase comprises the nucleotide sequence set forth in SEQ ID NO:3 comprising exons 1-13 of the mouse somatic ACE gene. In further aspects, the nucleotide sequence encodes a polypeptide comprising an amino acid sequence having at least 70%, 75%, 80%, 85%, 90%, 91%, 92%, 93%, 94%, 95%, 96%, 97%, 98%, 99% or more sequence identity to the amino acid sequence set forth in SEQ ID NO:4 corresponding to mouse somatic ACE.

In another embodiment, a transgenic animal is provided comprising at least one macrophage or macrophage-like cell having stably incorporated into its genome a polynucleotide sequence comprising a nucleotide sequence encoding a modulator of peptidase expression in macrophage and macrophage-like cells, wherein the modulator of peptidase expression inhibits transcription of a nucleotide sequence encoding the target peptidase, and wherein expression of the target peptidase is inhibited in the macrophage or macrophage-like cell. In other aspects, the target peptidase is a carboxypeptidase, particularly where the carboxypeptidase is an angiotensin-converting enzyme (ACE) or biologically active fragment thereof. In particular aspects, the transgenic animal is a mouse.

In one embodiment, the transgenic animals of the present invention are used to screen tumors and infectious agents to determine which are susceptible to ACE-mediated reduction in pathological effects. For example, tumors may be transplanted or pathologic agents administered into ACE over-expressing and control mice to see if the over-expressers show reduced tumor growth or infection.

In other embodiments, the transgenic animals of the present invention are used in epitope discovery and/or identification. Such transgenic animals are useful as tools to more efficiently identify epitopes involved in the immune response to tumors and infectious agents. Such methods of use include the ability to use transgenic animals of the present invention in in vivo screens alongside traditional epitope discovery methodologies.

Unless otherwise indicated, a transgenic animal comprises stable changes to the GERMLINE sequence. During the initial construction of the animal, chimeric animals (chimeras) are generated, in which only a subset of cells have the altered genome. Chimeras may then be bred to generate offspring heterozygous for the transgene. Male and female heterozygotes may then be bred to generate homozygous transgenic animals.

Transgenic animals can be produced by any suitable method known in the art, such as manipulation of embryos, embryonic stem cells, etc. Transgenic animals may be made through homologous recombination, where the endogenous locus is altered. Alternatively, a nucleic acid construct is randomly integrated into the genome. Vectors for stable integration include plasmids, retroviruses and other animal viruses, YACS, and the like.

Numerous methods for preparing transgenic animals are now known and others will likely be developed. See, e.g., U.S. Pats. Nos. 6,252,131; 6,455,757; 6,028,245; and 5,766,879, all incorporated herein by reference. Any method that produces a transgenic animal expressing a reporter gene following complementation or reconstitution is suitable for use in the practice of the present invention. The microinjection technique is particularly useful for incorporating transgenes into the genome without the accompanying removal of other genes.

In a further embodiment of the present invention, human macrophage and macrophage-like cells are provided that have been modified to either over-express at least one target peptidase or in which expression of at least one target peptidase has been disrupted, including, but not limited to, ACE. Methods for such modification of macrophage and macrophage-like cells are described in detail elsewhere herein. For example, in a particular embodiment of the invention, human macrophages are transfected with the human c-fms promoter to mediate over-expression of the ACE peptidase gene (see, e.g., Himes et al. (2001) J. Leukoc. Biol. 70:812-820). In another embodiment of the disclosure, such modified macrophage and macrophage-like cells are injected into patients in the treatment of various diseases and conditions as described elsewhere herein (e.g., into a cancer patient as a treatment for cancer including, but not limited to, melanoma or micrometastases of melanoma).

As used within these methods, the term “transfection” refers to the introduction of a nucleic acid sequence into the interior of a membrane enclosed space of a living cell, including introduction of the nucleic acid sequence into the cytosol of a cell as well as the interior space of a mitochondria, nucleus or chloroplast. “Transient transfection” refers to cases where exogenous nucleic acid is retained for a relatively short period of time, often when the nucleic acid does not integrate into the genome of a transfected cell, e.g., where episomal DNA is transcribed into mRNA and translated into protein. A cell has been “stably transfected” with a nucleic acid construct when the nucleic acid construct has been introduced inside the cell membrane and coding regions are capable of being inherited by daughter cells.

### Identification and Use of Modulators of Peptidase Expression in Macrophages

Additional understanding of how the over-expression or inhibition of expression of a peptidase such as ACE operates to enhance or suppress macrophage function may reveal ways of mimicking this phenomena in human macrophages through modification of the expression of peptidases besides ACE. Thus, the present invention also encompasses methods of identifying other modulators of peptidase expression in macrophages. Such modulators include, but are not limited to, compositions such as polypeptides, for example antibodies, small molecules, a nucleic acid molecule or other compounds which up-regulate or inhibit the activity of a peptidase in macrophage or macrophage-like cell. Such compounds may interact with cellular transcriptional elements and or the peptidase gene to increase or inhibit translation of the peptidase or such compounds may directly interact with the peptidase to augment or suppress its activity. In a particular embodiment such methods would screen for compounds which increase peptidase expression in macrophage and macrophage-like cells to enhance the immune function of such cells, but which will not increase peptidase expression in other host cells. In another embodiment such methods would screen for compounds which inhibit peptidase expression and/or activity in macrophage and macrophage-like cells to suppress the immune function of such cells, but which will not inhibit peptidase expression and/or activity in other host cells.

### EXPERIMENTAL

Now having described the embodiments of the compositions and methods for modulating macrophage activity, methods of making transgenic animals with modulated macrophage activity and methods for treating or preventing disease by modulating macrophage activity in general, the following examples describe certain embodiments of modulating macrophage activity, of transgenic animals having modulated macrophage activity and of treating cancer in a host by modulating macrophage activity in the host. While such embodiments are described in connection with Examples 1 and 2 and the corresponding text and figures, there is no intent to limit embodiments of modulating macrophage activity, methods of making transgenic animals with modulated macrophage activity and methods for treating or preventing disease by modulating macrophage activity to these descriptions. On the contrary, the intent is to cover all alternatives, modifications, and equivalents included within the spirit and scope of embodiments of the present disclosure.

As described elsewhere herein, embodiments of the present disclosure relate to the finding that modification of the expression of a target peptidase, including but not limited to ACE, in host macrophage and macrophage-like cells modifies macrophage function and can be used as a means to modify the natural immune defenses of a human or animal and to treat certain diseases and conditions. Specifically, mice in which macrophage and/or macrophage-like cells are modified to over-express the peptidase ACE are far more resistant to the growth of a tumor such as melanoma and exhibit resistance and/or immunity against viral infection. Although these findings suggest that the over expression of a peptidase such as ACE modifies/augments macrophage function and can be used as a means of augmenting the natural immune defenses of a human or animal to treat primary or metastatic tumors and viral infection, these findings also suggest that modulating (increasing or inhibiting) expression of a peptidase such as ACE modifies macrophage function and can be used as a means of modulating (enhancing or suppressing) the natural immune defenses of a human or animal to treat a variety of diseases and conditions associated with increased or decreased immune function, including cancer, viral and/or bacterial infection (including HIV infection), Alzheimer's disease, tissue transplant rejection, autoimmune diseases, and chronic inflammatory diseases.

### Example 1

To investigate the role of ACE in disease, a genetically modified mouse model was created in which the endogenous ACE gene has been modified by targeted homologous recombination. In this mouse, control of ACE expression was directed by a 6.7 kb c-fms promoter (SEQ ID NO:2). The promoter construct used in making ACE.10 mice contained important c-fms intron 2 regulatory elements that targets reporter gene expression to macrophages. (Himes et al. (2001) “A Highly Conserved c-fms Gene Intronic Element Controls Macrophage-Specific and Regulated Expression,” J. Leukoc. Biol., 70: 812-820). To our knowledge, our studies are the first to use the c-fms promoter to express elevated levels of a peptidase such as ACE in macrophage and macrophage-like cells.

### Creation and Testing of ACE 10 Mice

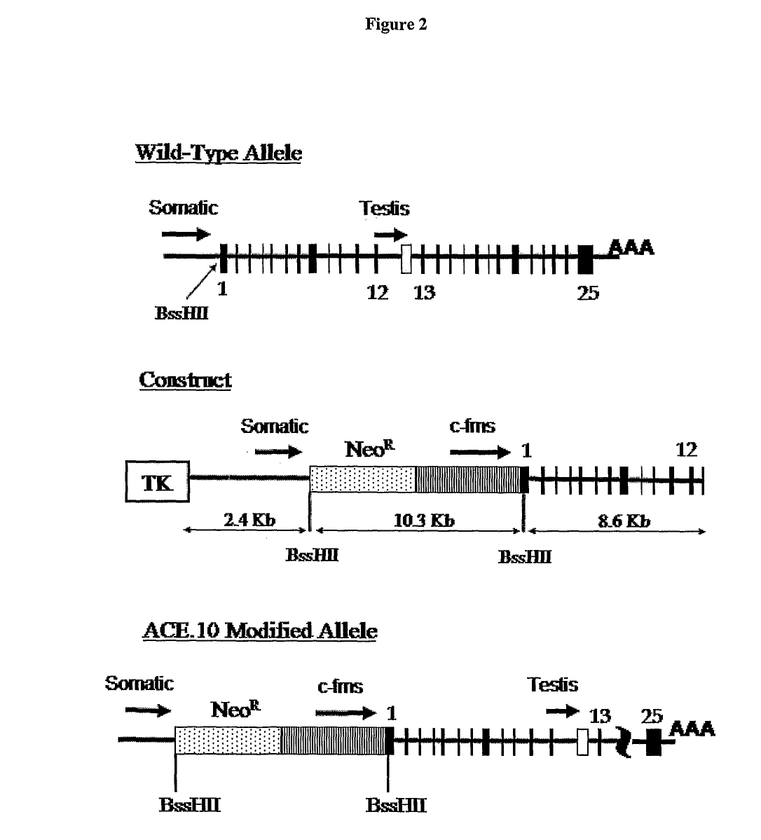

The strategy for creating ACE.10 mice was based on homologous recombination to place the c-fms promoter immediately 5′ of the ACE gene (see FIG. 2). The targeting construct was made and properly targeted ES cells were selected (see FIG. 2, described in more detail below). Homozygous mutant mice (called ACE 10/10) were first available for study in October, 2003. Because the c-fms promoter is specific for macrophage and macrophage-like cells, these modified mice do not express ACE in the vascular adventitial or endothelium. In addition, the kidneys of these animals are devoid of ACE activity. Despite this restricted ACE expression, the genetically modified mice (ACE 10/10 mice) have a normal blood pressure and renal function.

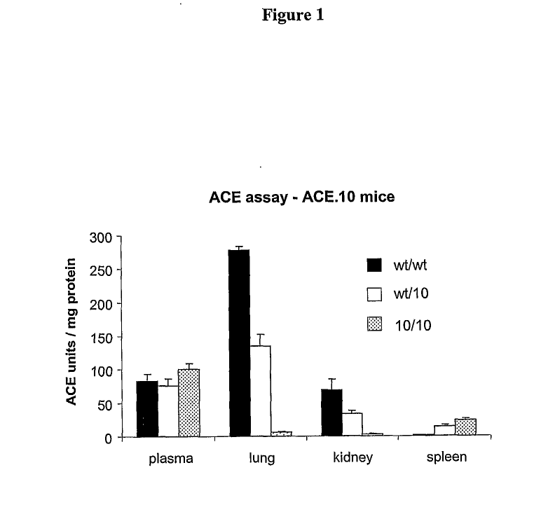

Wild-type (ACE wt/wt), heterozygous (ACE wt/10) and ACE 10/10 mice were sacrificed and individual tissue extracts were tested for ACE activity (FIG. 1). While a wild-type mouse has abundant pulmonary ACE, ACE 10/10 mice have a reduction of approximately 98% (WT: 277 vs. 6 U/mg for 10/10). Similarly, a wild-type mouse has abundant ACE in both endothelium and proximal tubular epithelium of the kidney. In contrast, ACE 10/10 mice have virtually no detectable ACE activity in the kidney. One organ in which the ACE 10/10 mice had increased ACE expression was the spleen in which there was 20-fold the enzyme activity found in wild-type mice.

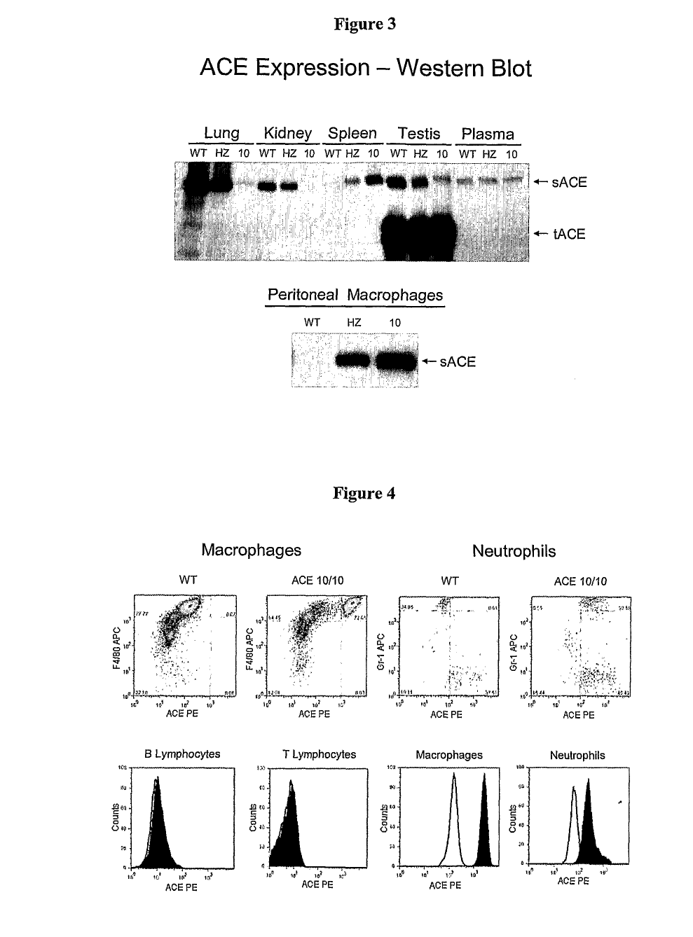

To understand ACE expression patterns in more detail, we performed immunostaining using an anti-ACE antibody. These data confirmed the enzyme activity/Western blot assays by showing ACE expression restricted to macrophages and macrophage lineage cells (FIG. 3).

To investigate the expression of ACE by hematopoietic tissues, FACS analysis was performed on white blood cells collected from the blood, spleen, or peritoneal washes (FIG. 4). Cells were stained with an anti-ACE antibody and antibodies to either CD3 (specific for T cell), B220 (B cells), CD11b (monocyte and macrophages), CD11c (dendritic cells), and GR-1 (neutrophils). FACS analysis was performed using two color labeling and standard procedures. In this assay, neutrophils were CD11b^{Intermed.}/GR-1^{Hi} while macrophages were CD11B^{Hi}/GR-1^{Dim}. For all assays, controls included identically prepared cells from wild-type mice and ACE 4/4 mice (somatic ACE null mice). The two control populations gave identical results. When cells were gated and compared for the level of ACE expression, no ACE expression was detected in the T cells, B cells or neutrophils of either strain of mice (FIG. 4). A high level of ACE expression, however, was detected in the monocytes/macrophages from ACE 10/10 mice, which was about 12-fold higher than that of the monocytes/macrophages from either the ACE 4/4 or wild-type mice based on mean channel fluorescence. There was some background staining of ACE 4/4 macrophages, which was non-specific. Thus, these data confirm our hypothesis that ACE 10/10 mice have ACE expression targeted to macrophages and macrophage lineage cells. ACE expression by such cells is substantially increased. This includes dendritic cells derived from the monocyte/macrophage lineage, which were also found to over-express ACE in ACE 10/10 mice.

The ACE 10/10 mice have a systolic blood pressure that averaged 100±8 mm Hg which was equivalent to that of wild-type mice (99±8 mm Hg). Renal function was examined by assessing the ability to concentrate urine after 24 h of water deprivation. ACE 10/10 mice achieved urinary concentrations averaging 3258±91 mOsm/kg H₂O, which was similar to that of wild-type mice (3470±183 mOsm/kg H₂O). GFR, as assessed with FITC-tagged inulin, (Qi et al. (2004) “Serial Determination of Glomerular Filtration Rate in Conscious Mice Using FITC-Inulin Clearance,” Am. J. Physiol. Renal Physiol., 286:F590-F596) was normal (319 μl/min vs 288 μl/min for control mice).

### ACE 10 Mice and Cancer

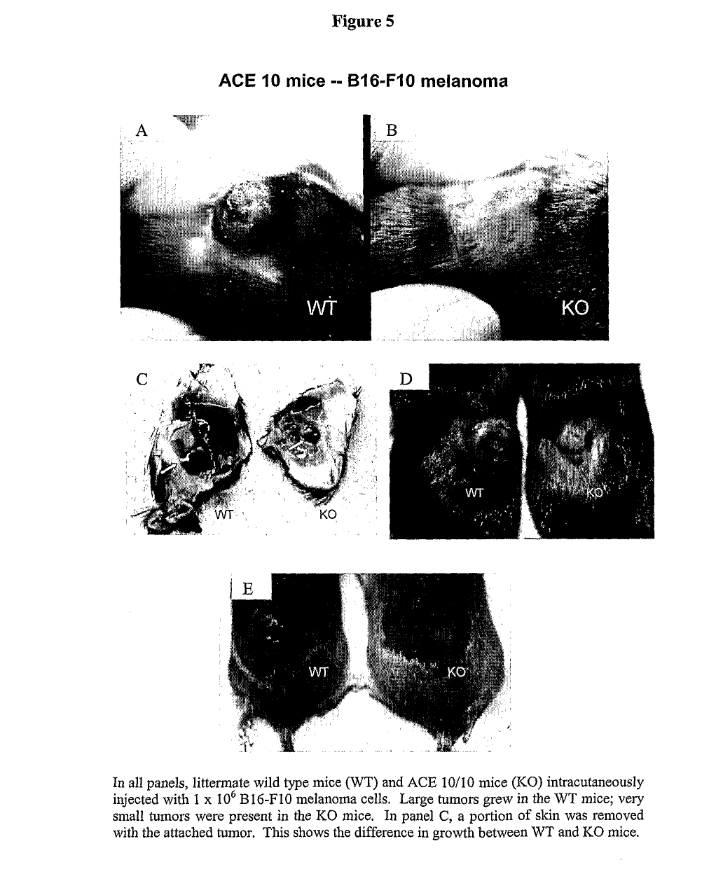

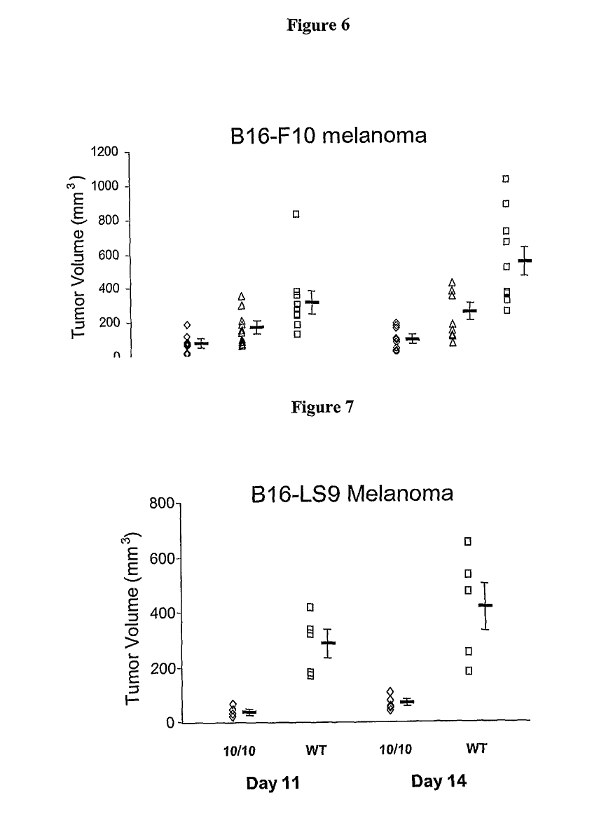

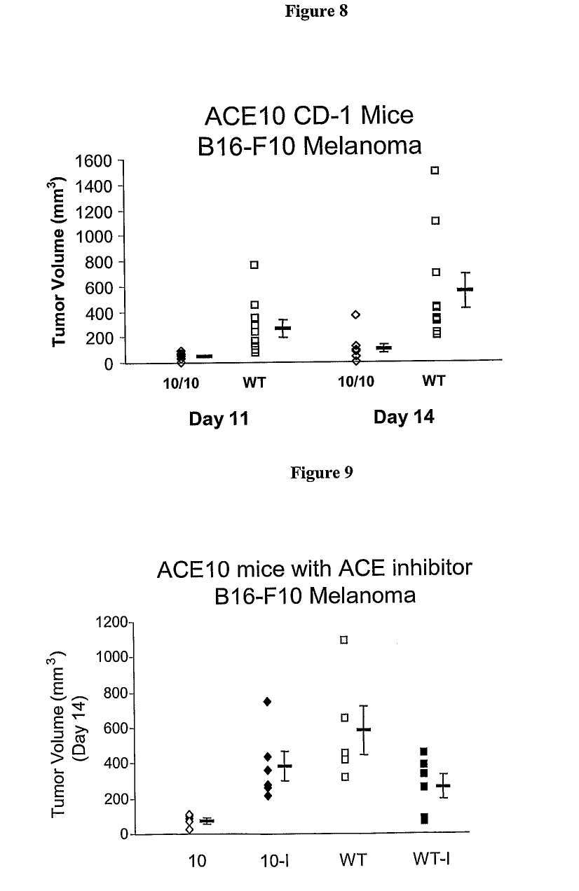

Recently, we studied the response of the ACE 10/10 mice to intradermal implantation of B16-F1 melanoma cells. In this model, littermate wild type or ACE 10/10 mice are intradermally (or subcutaneously) inoculated with 1×10⁶ melanoma cells. This is an aggressive tumor and within two weeks will form a large tumor nodule in a wild-type mouse. To our surprise, ACE 10/10 mice have far less tumor growth than wild-type mice. While wild-type animals repeatedly developed large, aggressive tumors, ACE 10/10 mice (macrophage ACE over expression) showed very little tumor growth. As seen in the pictures comprising FIG. 5, the difference is truly striking. FIG. 6 shows a quantification of the difference in tumor size, measured in volume, present in littermate wild type (WT) and ACE 10/10 mice (KO) measured 11 and 14 days after the injection of 1×10⁶ B16-F10 melanoma cells. We have performed this experiment many times and there is now no question but that the ACE 10/10 mice are far less susceptible to tumor growth than wild-type animals. Similar results were obtained following intradermal implantation of B16-LS9 melanoma cells in ACE 10/10 compared to wild type mice (FIG. 7), as well as in tumor growth between wild type and ACE 10/10 mice bred to a CD-1 (Swiss) outbred background (FIG. 8).

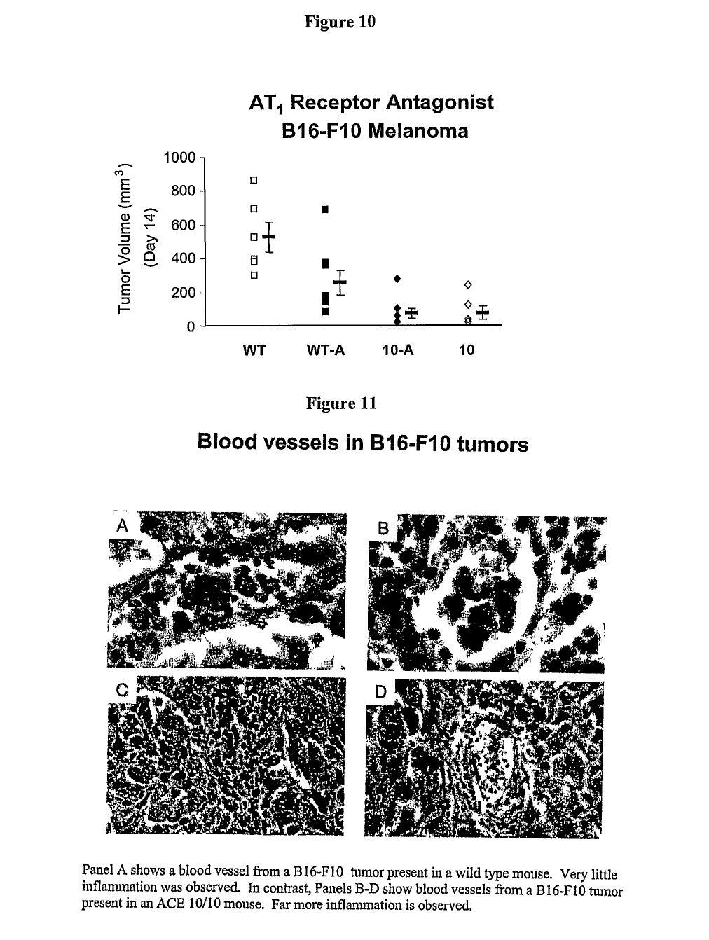

Quantitation of B16-F10 tumor growth in wild type and ACE 10/10 mice was also conducted between groups in which the ACE inhibitor captopril was also administered. Results indicated that in the presence of an ACE inhibitor, the ACE 10/10 were comparable to wild-type mice in terms of susceptibility to tumor growth (FIG. 9). This experiment establishes that it is the presence of ACE in the ACE 10/10 mice that is important in the observed effects on tumor growth compared to wild-type mice. By contrast, the treatment of wild type and ACE 10/10 with the AT1 receptor antagonist losartan does not inhibit the ability of the ACE 10/10 mice to resist melanoma (FIG. 10) The implication is that angiotensin II production is not critical to tumor resistance in ACE 10/10 mice.

Microscopic evaluation of the tumors present in wild-type mice and the small tumors present in the ACE 10/10 animals reveals, at least in part, why an animal with over expression of ACE in macrophages reacts so dramatically different from a wild-type mouse. This is demonstrated in FIG. 11 which shows blood vessels from the tumors present in wild-type mice and the tumors found in the ACE 10/10 mice. In wild-type mice, growth of the B16-F10 tumor is accompanied by blood vessels without any real evidence of an immune response. In contrast, the blood vessels from tumors taken from the ACE 10/10 mice are loaded with inflammatory cells. Immunohistochemical staining of these cells revealed abundant macrophages and T-cells. Histology of tumor blood vessels in melanoma tumors from wild type and ACE 10/10 mice indicated that more white cells were present in the ACE 10/10 blood vessels, that these white cells are CD11b positive (which is a monocyte marker), and that these white cells express abundant ACE as determined by staining with anti-ACE antibody. Immunohistochemical results also showed that phagocytic cells within the tumor express ACE, that these phagocytic cells may phagocytize portions of the tumor cells, and that such phagocytic cells are macrophages as indicated by positive staining for F4/80. To summarize, ACE 10/10 mice are markedly resistant to the growth of an aggressive melanoma cell line. This appears correlated with a more vigorous immune response in the ACE 10/10 mice.

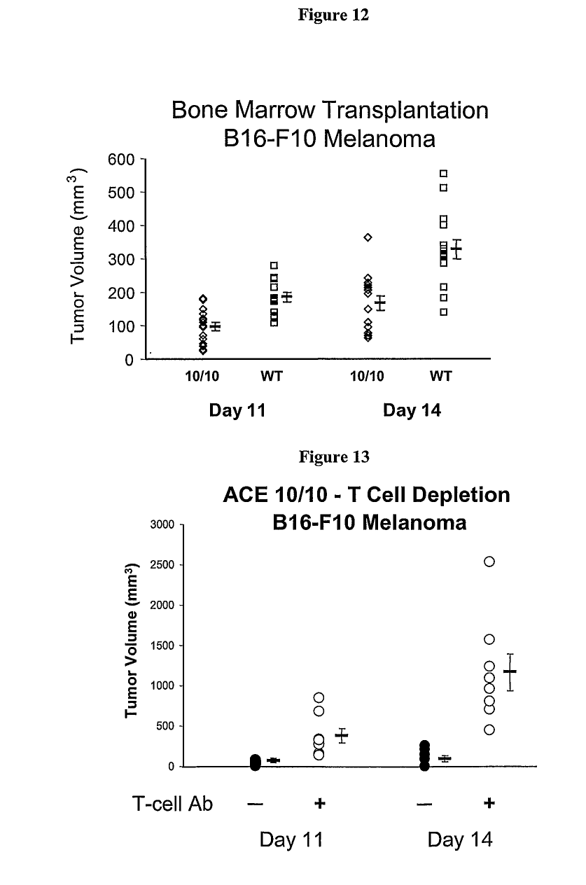

The transferability of the phenotype of ACE 10/10 mice was also investigated. Wild type mice were lethally irradiated and then rescued by bone marrow transplantation from either ACE 10/10 or wild type mice. After 8 weeks to restore the marrow, the mice were challenged by intradermal injection of B16-F10 melanoma cells. Two weeks later, tumor size was measured. Mice receiving bone marrow from ACE 10/10 mice were more resistant to the tumor (FIG. 12) indicating that this phenotype is transferable with bone marrow transplantation.

It is possible that the over expression of a peptidase such as ACE in macrophages may change the phenotype of these cells in terms of either cytokine production, motility or the generation of reactive oxygen species (ACE produces angiotensin II which stimulates reactive oxygen generation). However, it is believed that the primary explanation for the phenomenon centers around the fact that ACE is a peptidase. One of the critical functions of macrophages is to process immune reactive peptides, which are finally placed on the macrophage cell surface and act as general stimuli for both B and T cells. As shown in FIG. 13, ACE 10/10 mice were injected with anti-CD4 and anti-CD8 monoclonal antisera (indicated by +), which eliminates T cells but does not eliminate monocytes or macrophages. Eliminating T cells eliminates the resistance of ACE 10/10 mice to tumor growth. This indicates that ACE 10/10 mice have an enhanced immune response that plays a role in providing resistance to tumor growth.

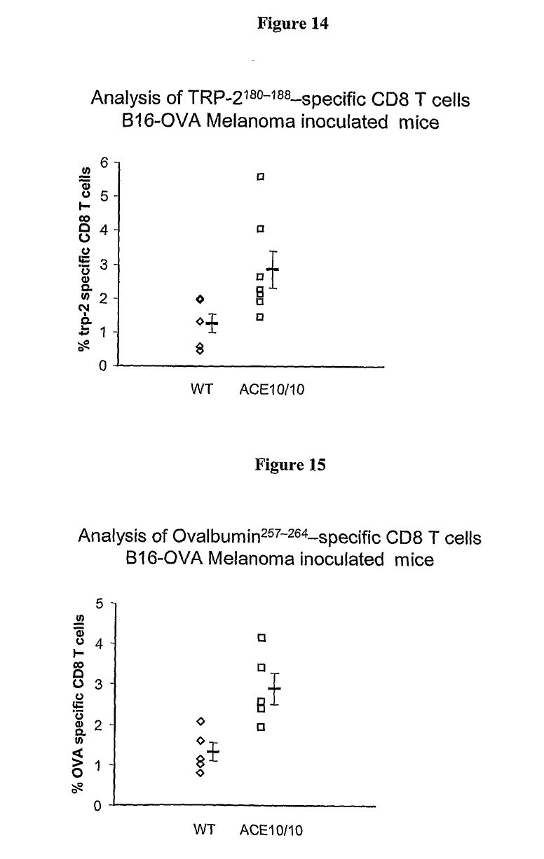

T cell immune response to melanoma in the ACE 10/10 mice was also investigated by cutaneously injecting control and ACE 10/10 mice with a B16 melanoma that was engineered to express ovalbumin. This was called B16-Ova. Two weeks after inoculation, blood CD8 T cells were measured using tetramer staining. We measured two different tetramers: TRP-2¹⁸⁰⁻¹⁸⁸ and Ovalbumin²⁵⁷⁻²⁶⁴. The TRP-2 peptide is known to be an immunodominant peptide of B16 melanomas, while the ovalbumin peptide is the immunodominant peptide for that protein. In both instances, there was increased CD8 T cell tetramer binding in the ACE 10/10 mice (FIGS. 14 and 15). This is evidence of a greater T cell immune response to melanoma in the ACE 10/10 mice.

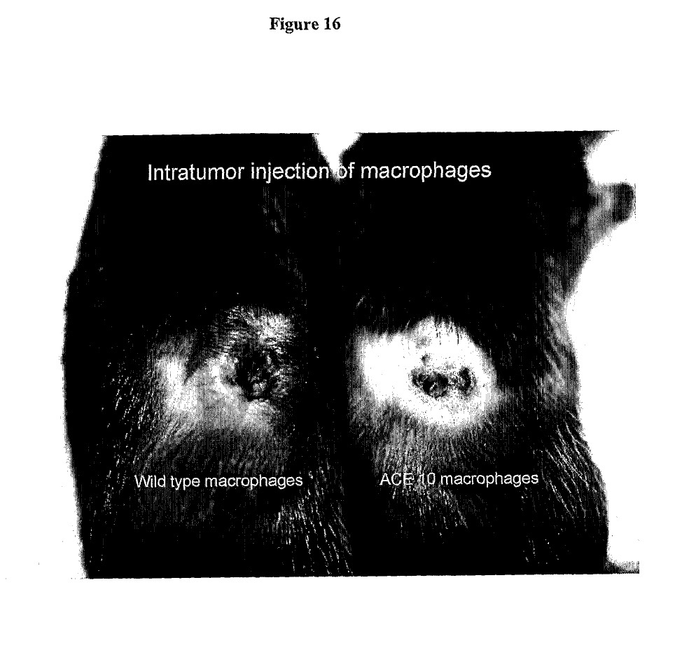

Thus it is believed that the modification of a macrophage by the over expression of a peptidase such as ACE may change the ability of a macrophage to process peptides thus changing the immunoreactivity of the macrophage. It is believed that such “super” macrophages are better able to process peptides and initiate an immune response. Such a response is consistent with additional results in which one week after wild type mice were injected intradermally with B16-F10 melanoma cells, the tumor nodules were injected with 2 million wild type or ACE 10/10 macrophages. The tumors were then allowed to grow for 12 more days. Injection of ACE 10/10 macrophages was more effective in reducing tumor size than macrophages from a wild-type mouse (FIG. 16).

### Methods for Example 1

Creation of ACE.10 homozygous mutant mice. A 10.7 kb fragment of mouse genomic DNA was cloned from a mouse CC1.2 ES cell library (FIG. 2). This contained 2.4 kb of the somatic ACE promoter, the somatic ACE transcription start site and 8.6 kb of genomic sequence encompassing somatic ACE exons 1-13 (SEQ ID NO:3). A neomycin cassette (called KT3NP4) was inserted into a unique BssH II restriction site located within the 5′ untranslated region of somatic ACE. A 7.2 kb fragment of DNA encoding the mouse cams promoter (SEQ ID NO:2), the first and second c-fms exons and the first and second introns was positioned immediately 3′ to the neomycin cassette within the targeting vector. This construct was chosen since previous work has demonstrated that the c-fms intron II contains a DNase I hypersensitive site required for expression of a reported gene macrophage cell lines. (Lichanska et al. (1999) “Differentiation of the Mononuclear Phagocyte System During Mouse Embryogenesis: The Role of Transcription Factor PU.1,” Blood, 94:127-138; Himes et al. (2001) “A Highly Conserved c-fms Gene Intronic Element Controls Macrophage-Specific and Regulated Expression,” J. Leukoc. Biol., 70: 812-820). The c-fms promoter had previously been modified so that the translation start site, normally present in exon II was no longer functional. Our strategy positions the neomycin resistance cassette to block any influence of the endogenous somatic ACE promoter on ACE gene transcription. Our strategy also positions the c-fms promoter in a position to control the transcription of the gene encoding somatic ACE.

The ACE.10 targeting construct was linearized and electroporated into R1 ES cells derived from a 129/SVx129/SvJ F1 embryo. Individual ES cell clones were screened for targeted homologous recombination using a combination of PCR and genomic Southern blot analysis. The generation of chimeric mutant mice was performed as previously described. Chimeric mice were mated to C57BL/6 mice to generate F1 mice. Heterozygous F1 mice were bred to create F2 offspring of wild-type (WT/WT), heterozygous (WT/10) and homozygous ACE.10 (ACE 10/10) mice. All studies were performed on F2 or F3 generation litters generated from the breeding of heterozygous animals. Age and gender matched littermate controls were used in all studies. Animal procedures were approved by institutional animal care and use committee and were supervised by the Emory University Division of Animal Research.

Genotyping of mice. Genomic DNA was obtained through tail clipping. Four primers were used for PCR genotyping. The wild-type ACE allele was detected with the primers 5′ CTAGCTTCCTCTGAGAGAGCC 3′ (SEQ ID NO:5) and 5′ CCTCGGCACTCGAGTTATAGC 3′ (SEQ ID NO:6) which amplified a band of 423 bp, while the targeted allele was detected with the primers 5′ GCAGGTCATGGTTATCTGGTG 3′ (SEQ ID NO:7) (neo cassette) and 5′ CTGAGGAAGTGCAGGACTTAC 3′ (SEQ ID NO:8) (c-fms) which amplified a band of 400 bp.

ACE activity assay. Cardiac puncture was performed on anesthetized mice to collect blood in heparinized tubes. Plasma was obtained by centrifugation of blood samples at 4° C. for 10 minutes at 2000 g. Animals were then sacrificed and tissue samples were collected. Individual tissues were briefly homogenized at low speed in ACE homogenization buffer (50 mmol/l HEPES, pH 7.4, 150 mmol/l NaCl, 25:mol/l ZnCl₂, and 1 mmol/1 PMSF) lacking detergent. These homogenates were centrifuged at 10,000 g and the supernatant discarded. The pellets were then resuspended in ACE homogenization buffer containing 0.5% Triton X-100 and vigorously re-homogenized. The tissue homogenates were again spun at 10,000 g and supernatants were used for ACE activity measurement. Due to the small size of atrial tissues, a small hand-held motorized glass-pestle homogenizer was used following the same procedure. ACE activity was measured using the ACE-REA kit from American Laboratory Products Company, Ltd. (Alpco, Windham, N.H.). ACE activity assay was performed following the kit instructions and activity was defined as that inhibited by captopril. Protein concentration was measured using BCA Protein Assay Reagent kit (Pierce Rockford, Ill.). Tissue ACE activity was calculated as unit per microgram protein.

Western blotting, collagen staining and immunohistochemistry. For Western blot, tissue homogenates were prepared as described for the ACE activity assay. Protein samples (20:g per lane) were separated on an 8% SDS gel and transferred to a nitrocellulose membrane. The membrane was blotted using a rabbit polyclonal anti-mouse ACE antibody (Lichanska et al. (1999) “Differentiation of the Mononuclear Phagocyte System During Mouse Embryogenesis: The Role of Transcription Factor PU.1,” Blood, 94:127-138) and exposed to x-ray film using the enhanced chemiluminescence method.

For histologic analysis, tissue samples were taken at euthanasia and preserved in 10% neutral-buffered formalin. Lung tissues were infused with formalin through the trachea. Tissues were then embedded in paraffin using standard procedures. Sections were then stained for hematoxylin and eosin using standard techniques. For immunohistochemistry, both ACE 10/10 and wild-type tissues were placed on a single slide. Immunohistocchemical detection of ACE was performed as previously described (Lichanska et al. (1999) “Differentiation of the Mononuclear Phagocyte System During Mouse Embryogenesis: The Role of Transcription Factor PU.1,” Blood, 94:127-138).

Blood pressure. Systolic blood pressure was measured in conscious mice using a Visitech Systems BP2000 automated tail cuff system (Apex, N.C.) as previously described (Powell et al. (1989), “Inhibitors of Angiotensin-Converting Enzyme Prevent Myointimal Proliferation After Vascular Injury,” Science, 245:186-188). Mice were trained in the apparatus for five days before data were collected. The blood pressure of an animal was the average of 80 measurements over an additional four days.

Blood electrolytes and urine osmolality. For blood electrolytes, mice were anesthetized with ketamine (125 mg/kg) and xylazine (12.5 mg/kg). Arterial blood was collected from the carotid artery into Microtainer tubes coated with lithium heparin (Becton Dickinson, N.J.). Blood samples were analyzed immediately for electrolytes using a NOVA CRT 16 electrolyte analyzer.

Spot urine samples were collected before and after 24 hours of water deprivation. Urine samples were spun at 5500 g to precipitate particulates. Urine osmolality was determined using a Wescat 5500 Vapor Pressure Osmometer (Wescor Inc).

FACS analysis. Fresh peripheral blood was collected by cardiac puncture in Microtainer tubes that contained EDTA at a final concentration of 1 mM. Spleens were removed and washed with DMEM, and then were minced into fragments and incubated in lysis buffer (0.2 mg/ml dispase, 0.1% DNase and 1.6 mg/ml collagenase) for 15 minutes. After centrifugation, the pellet was washed in PBS and then resuspended in PBS. Peritoneal cells were obtained through peritoneal lavage with PBS.

All cell suspensions were incubated with antibody for 30 min in PBS containing 0.5% BSA, 2 mM EDTA and 0.1% sodium azide. Monoclonal antibodies included a fluorescein isothiocyanate (FITC) labelled antibody to mouse CD3 (dilution of 1:100), a peridin-chlorophyll protein (PerCP) labelled antibody to B220 (dilution 1:100), a biotin labelled antibody to CD11b (dilution 1:100) and an FITC labelled antibody to GR-1 (dilution 1:100). We also used streptavidin-allophycocyanin (SA-APC) (dilution 1:100) for the CD11b determination. These antibodies were purchased from BD Pharmingen. We used a 1:250 dilution of a rabbit polyclonal anti-mouse ACE antiserum prepared in our lab. This was followed by a 20 min incubation on ice with a 1:400 dilution of phycoerythrin labelled goat anti-rabbit Ig (BD Pharmingen). Non-immune rabbit serum was used as a primary antibody control. After antibody addition binding, cells were washed 3× with PBS. Any red blood cells were lysed with lysis buffer purchased from SIGMA Chemical Co. After this, samples were washed 2× in PBS. All samples were analyzed on FASCAN using Cellquest software. 50000 events were collected of each specimen.

B16-F10 tumor protocol. B16-F10 melanoma cell line was obtained from ATCC (American Type Culture Collection, Manassas, Va., USA). Cells were cultured in DMEM supplemented with 4 mM L-glutamine, 4.5 g/l glucose, 10% FBS, and antibiotics under humidified air with 5% CO₂ at 37° C. Before use, the melanoma cell monolayer in culture was washed and detached twice with PBS (Ca²⁺ and Mg²⁺ free) containing 0.25% trypsin and 0.03% EDTA and then pelleted by brief centrifugation at 100 g. The supernatant was removed, cell pellets were resuspended in PBS, and the cell number was counted.

Mice aged 8 to 12 weeks received exponentially growing cells (1×10⁶) interdermally into the dorsal skin using a disposable 27-gauge needle under anesthesia (28 mg/kg avertin, intraperitoneally). Measurements of tumor size (longest and shortest diameters) were done using calipers at the 11th and 14th day, and body weight was measured at the day of inoculation (Day 0) and at harvesting (the 14th day), respectively. Tumor volume was calculated by the formula for a prolate spheroid according to the equation (L [longest diameter] H S2 [shortest diameter] H 0.52)=tumor volume.

### Example 2

In this example, the immune response of ACE 10/10 mice in a model of chronic viral infection was studied. Mouse lymphocytic choriomeningitis virus (LCMV) is an arenavirus, and is often used as a model of chronic viral infection (Barber et al. (2006) Nature 439:682-7; Wherry et al. (2005) J. Virol. 79:8960-8968). Typically, LCMV is cleared by healthy wild-type mice during an acute-infection phase, but there are several highly invasive laboratory-derived LCMV strains that cause long-term infection in blood and multiple organs. One such LCMV strain is termed Clone 13.

We infected wild-type (WT) and ACE 10/10 mice (KO) with Clone 13 and then studied the T cell response using tetramer staining. After the initial phase of infection, ACE 10/10 mice showed a consistent increase in the blood T cell frequency for the GP33 peptide (FIG. 17). The GP33 peptide is one of the major antigenic peptides of LCVM Clone 13 infection, and has even been used as a means of vaccinating against Clone 13 (see, e.g., Wherry et al. (2005) J. Virol. 79:8960-8968). Thus, ACE 10/10 mice have higher levels of CD8 T cells typically associated with immunity against Clone 13 LCMV. Further, ACE 10/10 mice cleared the Clone 13 virus from the blood dramatically faster than wild-type mice (FIG. 18, ACE 10/10 mice are marked KO). These data support our hypothesis that the ACE 10/10 mice have an enhanced immune response, and that this technology may be applicable to chronic viral infections in humans.

### Discussion for Examples 1 and 2

In summary, we successfully prepared a mouse model wherein the mice over-express a peptidase (ACE) in macrophages and macrophage derived cells. The over expression of ACE was achieved using the c-fms promoter. In this novel line of mice, termed, ACE 10/10, there is no ACE produced by endothelium, glomerular mesangial cells or renal tubular epithelium. Plasma ACE was no different from wild-type mice. Blood pressure and renal development were normal. Furthermore, this novel line of mice showed marked resistance to the growth of a tumor such as the B16-F10 melanoma cell line, as well as enhanced immune response following exposure to LCMV. Study of this model strongly suggests that the over expression of a peptidase such as ACE by macrophages and macrophage related cells is a novel cancer therapy due to the activation of macrophages and the stimulation of natural immunity against tumor cells. These findings also suggest that modulating (increasing or inhibiting) expression of a peptidase such as ACE modifies macrophage function and can be used as a means of modulating (enhancing or suppressing) the natural immune defenses of a human or animal to treat a variety of diseases and conditions associated with increased or decreased immune function, including cancer, viral and/or bacterial infection (including HIV infection), Alzheimer's disease, tissue transplant rejection, autoimmune diseases, and chronic inflammatory diseases.

It should be emphasized that the above-described embodiments of the present disclosure are merely possible examples of implementations, and are set forth only for a clear understanding of the principles of the disclosure. Many variations and modifications may be made to the above-described embodiments of the disclosure without departing substantially from the spirit and principles of the disclosure. All such modifications and variations are intended to be included herein within the scope of this disclosure and protected by the following claims.

It must be noted that, as used in the specification and the appended claims, the singular forms “a,” “an,” and “the” include plural referents unless the context clearly dictates otherwise.

All publications and patent applications mentioned in the specification are indicative of the level of those skilled in the art to which this invention pertains. All publications and patent applications are herein incorporated by reference to the same extent as if each individual publication or patent application was specifically and individually indicated to be incorporated by reference.

Embodiments of the present disclosure employ, unless otherwise indicated, conventional techniques of synthetic organic chemistry, cell biology, cell culture, biochemistry, molecular biology, transgenic biology, microbiology, recombinant DNA, immunology, and the like, which are within the skill of the art. Such techniques are explained fully in the literature (See, e.g., Molecular Cloning, A Laboratory Manual, 2nd Ed., ed. by Sambrook, Fritsch and Maniatis (Cold Spring Harbor Laboratory Press: 1989); DNA Cloning, Volumes I and II (D. N. Glover ed., 1985); Oligonucleotide Synthesis (M. J. Gait ed., 1984); U.S. Pat. No. 4,683,195; Nucleic Acid Hybridization (B. D. Hames & S. J. Higgins eds. 1984); Transcription and Translation (B. D. Hames & S. J. Higgins eds. 1984); Culture of Animal Cells (R. I. Freshney, Alan R. Liss, Inc., 1987); Immobilized Cells and Enzymes (IRL Press, 1986); B. Perbal, A Practical Guide to Molecular Cloning (1984); the treatise, Methods in Enzymology (Academic Press, Inc., N.Y.); Gene Transfer Vectors for Mammalian Cells (J. H. Miller and M. P. Calos eds., 1987, Cold Spring Harbor Laboratory); Methods in Enzymology, Vols. 154 and 155 (Wu et al. eds.), Immunochemical Methods in Cell and Molecular Biology (Mayer and Walker, eds., Academic Press, London, 1987); Handbook of Experimental Immunology, Volumes I-IV (D. M. Weir and C. C. Blackwell, eds., 1986); Manipulating the Mouse Embryo (Cold Spring Harbor Laboratory Press, Cold Spring Harbor, N.Y., 1986).

Although the foregoing invention has been described in some detail by way of illustration and example for purposes of clarity of understanding, it will be obvious that certain changes and modifications may be practiced within the scope of the appended claims.

1. A method for increasing the activity of a target peptidase in a macrophage or macrophage-like cell, said method comprising introducing into said cell a nucleotide construct comprising a polynucleotide sequence operably linked to at least one promoter that is functional in said cell, wherein said polynucleotide comprises a nucleotide sequence encoding said target peptidase, wherein at least one of said promoters increases transcription of said nucleotide sequence encoding said target peptidase, and wherein said target peptidase is over-expressed in said macrophage or macrophage-like cell.

2. The method of claim 1, wherein said nucleotide construct further comprises a nucleotide sequence encoding a selectable marker.

3. The method of claim 2, wherein said macrophage or macrophage-like cell has been stably transfected with said nucleotide construct.

4. The method of claim 3, wherein said target peptidase is a carboxypeptidase.

5. The method of claim 4, wherein said carboxypeptidase comprises an amino acid sequence selected from the group consisting of:
a) the amino acid sequence of angiotensin-converting enzyme set forth in SEQ ID NO:1;
b) an amino acid sequence having at least 80% sequence identity to the amino acid sequence set forth in SEQ ID NO: 1, wherein said amino acid sequence encodes a peptidase that cleaves angiotensin I and converts angiotensin I into angiotensin II; and
c) an amino acid sequence having at least 90% sequence identity to the amino acid sequence set forth in SEQ ID NO: 1, wherein said amino acid sequence encodes a peptidase that cleaves angiotensin I and converts angiotensin I into angiotensin II.

6. The method of claim 5, wherein said promoter is a c-fms promoter.

7. A macrophage or macrophage-like cell having stably incorporated into its genome a polynucleotide sequence operably linked to at least one promoter that is functional in said cell, wherein said polynucleotide comprises a nucleotide sequence encoding a target peptidase, wherein at least one of said promoters increases transcription of said nucleotide sequence encoding said target peptidase, and wherein said target peptidase is over-expressed in said macrophage or macrophage-like cell.

8. The macrophage or macrophage-like cell of claim 7, wherein said target peptidase is a carboxypeptidase.

9. The macrophage or macrophage-like cell of claim 8, wherein said carboxypeptidase comprises an amino acid sequence selected from the group consisting of:
a) the amino acid sequence of angiotensin-converting enzyme set forth in SEQ ID NO:1;
b) an amino acid sequence having at least 80% sequence identity to the amino acid sequence set forth in SEQ ID NO: 1, wherein said amino acid sequence encodes a peptidase that cleaves angiotensin I and converts angiotensin I into angiotensin II; and
c) an amino acid sequence having at least 90% sequence identity to the amino acid sequence set forth in SEQ ID NO: 1, wherein said amino acid sequence encodes a peptidase that cleaves angiotensin I and converts angiotensin I into angiotensin II.

10. A method for enhancing the immune response of a patient in need thereof, said method comprising introducing into said patient a stably transfected macrophage or macrophage-like cell according to claim 7.

11. A method for treating cancer in a patient in need thereof, said method comprising introducing into said patient a stably transfected macrophage or macrophage-like cell according to claim 7.

12. The method of claim 11, wherein said cancer is metastatic or micrometastatic cancer.

13. The method of claim 11, wherein said cancer is melanoma or micrometastases of melanoma.

14. A method for treating pathogenic infection in a patient in need thereof, said method comprising introducing into said patient a stably transfected macrophage or macrophage-like cell according to claim 7.

15. The method of claim 14, wherein said pathogenic infection is a bacterial infection.

16. The method of claim 15, wherein said bacterial infection is caused by caused by Escherichia coli, Helicobacter pylori, Listeria monocytogenes, Salmonella typhimurium, Shigella flexneri, or Mycobacterium tuberculosis.

17. The method of claim 14, wherein said pathogenic infection is a viral infection.

18. The method of claim 17, wherein said viral infection is caused by a human immunodeficiency virus, a hepatitis virus, or a herpes virus.

19. A method for treating Alzheimer's disease in a patient in need thereof, said method comprising introducing into said patient a stably transfected macrophage or macrophage-like cell according to claim 7.

20. The method of claim 10, wherein said target peptidase is a carboxypeptidase.

21. The method of claim 20, wherein said carboxypeptidase comprises an amino acid sequence selected from the group consisting of:
a) the amino acid sequence of angiotensin-converting enzyme set forth in SEQ ID NO:1;
b) an amino acid sequence having at least 80% sequence identity to the amino acid sequence set forth in SEQ ID NO: 1, wherein said amino acid sequence encodes a peptidase that cleaves angiotensin I and converts angiotensin I into angiotensin II; and
c) an amino acid sequence having at least 90% sequence identity to the amino acid sequence set forth in SEQ ID NO: 1, wherein said amino acid sequence encodes a peptidase that cleaves angiotensin I and converts angiotensin I into angiotensin II.

22. A transgenic animal comprising at least one macrophage or macrophage-like cell according to claim 7.

23. The transgenic animal of claim 22, wherein said target peptidase is a carboxypeptidase.

24. The transgenic animal of claim 23, wherein said carboxypeptidase is an angiotensin-converting enzyme or biologically active fragment thereof.

25. An expression vector comprising a nucleotide construct comprising a polynucleotide sequence operably linked to at least one promoter that is functional in a macrophage or macrophage-like cell, wherein said polynucleotide comprises a nucleotide sequence encoding a target peptidase, wherein at least one of said promoters increases transcription of said nucleotide sequence encoding said target peptidase.

26. The expression vector of claim 25, wherein said nucleotide construct further comprises a nucleotide sequence encoding a selectable marker.

27. The expression vector of claim 25, wherein said target peptidase is a carboxypeptidase.

28. The expression vector of claim 27, wherein said carboxypeptidase comprises an amino acid sequence selected from the group consisting of:
a) the amino acid sequence of angiotensin-converting enzyme set forth in SEQ ID NO:1;
b) an amino acid sequence having at least 80% sequence identity to the amino acid sequence set forth in SEQ ID NO: 1, wherein said amino acid sequence encodes a peptidase that cleaves angiotensin I and converts angiotensin I into angiotensin II; and
c) an amino acid sequence having at least 90% sequence identity to the amino acid sequence set forth in SEQ ID NO: 1, wherein said amino acid sequence encodes a peptidase that cleaves angiotensin I and converts angiotensin I into angiotensin II.

29. The expression vector of claim 25, wherein said promoter is a c-fms promoter.

30. A method for inhibiting the activity of a target peptidase in a macrophage or macrophage-like cell, said method comprising introducing into said cell a nucleotide construct comprising a polynucleotide sequence operably linked to at least one promoter that is functional in said cell, wherein said polynucleotide comprises a nucleotide sequence encoding a modulator of peptidase expression in macrophage and macrophage-like cells, wherein said modulator of peptidase expression inhibits transcription of a nucleotide sequence encoding the target peptidase.

31. The method of claim 30, wherein said modulator of peptidase expression is an siRNA or an antisense polynucleotide.

32. The method of claim 30, wherein said nucleotide construct further comprises a nucleotide sequence encoding a selectable marker.

33. The method of claim 32, wherein said macrophage or macrophage-like cell has been stably transfected with said nucleotide construct.

34. The method of claim 33, wherein said target peptidase is a carboxypeptidase.

35. The method of claim 34, wherein said carboxypeptidase comprises an amino acid sequence selected from the group consisting of:
a) the amino acid sequence of angiotensin-converting enzyme set forth in SEQ ID NO:1;
b) an amino acid sequence having at least 80% sequence identity to the amino acid sequence set forth in SEQ ID NO: 1, wherein said amino acid sequence encodes a peptidase that cleaves angiotensin I and converts angiotensin I into angiotensin II; and
c) an amino acid sequence having at least 90% sequence identity to the amino acid sequence set forth in SEQ ID NO: 1, wherein said amino acid sequence encodes a peptidase that cleaves angiotensin I and converts angiotensin I into angiotensin II.

36. A macrophage or macrophage-like cell having stably incorporated into its genome a polynucleotide sequence comprising a nucleotide sequence encoding a modulator of peptidase expression in macrophage and macrophage-like cells, wherein said modulator of peptidase expression inhibits transcription of a nucleotide sequence encoding the target peptidase, and wherein expression of said target peptidase is inhibited in said macrophage or macrophage-like cell.

37. The macrophage or macrophage-like cell of claim 36, wherein said target peptidase is a carboxypeptidase.

38. The macrophage or macrophage-like cell of claim 37, wherein said carboxypeptidase comprises an amino acid sequence selected from the group consisting of:
a) the amino acid sequence of angiotensin-converting enzyme set forth in SEQ ID NO:1;
b) an amino acid sequence having at least 80% sequence identity to the amino acid sequence set forth in SEQ ID NO: 1, wherein said amino acid sequence encodes a peptidase that cleaves angiotensin I and converts angiotensin I into angiotensin II; and
c) an amino acid sequence having at least 90% sequence identity to the amino acid sequence set forth in SEQ ID NO: 1, wherein said amino acid sequence encodes a peptidase that cleaves angiotensin I and converts angiotensin I into angiotensin II.

39. A transgenic animal comprising at least one macrophage or macrophage-like cell according to claim 36.

40. The transgenic animal of claim 39, wherein said target peptidase is a carboxypeptidase.

41. The transgenic animal of claim 40, wherein said carboxypeptidase wherein said carboxypeptidase is an angiotensin-converting enzyme or biologically active fragment thereof.

42. The transgenic animal of claim 41, wherein said animal is a mouse, wherein said peptidase expression is directed by a c-fms promoter sequence, and wherein said nucleotide sequence encodes a polypeptide comprising an amino acid sequence selected from the group consisting of:
a) the amino acid sequence of mouse somatic angiotensin-converting enzyme set forth in SEQ ID NO:4;
b) an amino acid sequence having at least 80% sequence identity to the amino acid sequence set forth in SEQ ID NO:4, wherein said amino acid sequence encodes a peptidase that cleaves angiotensin I and converts angiotensin I into angiotensin II; and
c) an amino acid sequence having at least 90% sequence identity to the amino acid sequence set forth in SEQ ID NO:4, wherein said amino acid sequence encodes a peptidase that cleaves angiotensin I and converts angiotensin I into angiotensin II.

43-51. (canceled)
